(19) United States
(12) Patent Application Publication
(10) Pub. No.: US 2021/0315264 A1
(43) Pub. Date: Oct. 14, 2021

(54) SMOKING SUBSTITUTE CONSUMABLE

(71) Applicant: Nerudia Limited, Liverpool (GB)

(72) Inventors: Chris LORD, Liverpool (GB); Kate FERRIE, Liverpool (GB); Edward Ross SHENTON, Liverpool (GB)

(21) Appl. No.: 17/243,091

(22) Filed: Apr. 28, 2021

Related U.S. Application Data
(63) Continuation of application No. PCT/EP19/79181, filed on Oct. 25, 2019.
Application No. PCT/EP19/79181 is a continuation of application No. PCT/EP19/79161, filed on Oct. 25, 2019.
Application No. PCT/EP19/79161 is a continuation of application No. PCT/EP19/79183, filed on Oct. 25, 2019.
Application No. PCT/EP19/79183 is a continuation of application No. PCT/EP19/79200, filed on Oct. 25, 2019.
Application No. PCT/EP19/79200 is a continuation of application No. PCT/EP19/79272, filed on Oct. 25, 2019.
Application No. PCT/EP19/79272 is a continuation of application No. PCT/EP19/79197, filed on Oct. 25, 2019.
Application No. PCT/EP19/79197 is a continuation of application No. PCT/EP19/79208, filed on Oct. 25, 2019.
Application No. PCT/EP19/79208 is a continuation of application No. PCT/EP19/79227, filed on Oct. 25, 2019.

(30) Foreign Application Priority Data
Oct. 29, 2018 (GB) 1817534.9
Oct. 29, 2018 (GB) 1817535.6
Oct. 29, 2018 (GB) 1817546.3
Oct. 29, 2018 (GB) 1817550.5
Oct. 29, 2018 (GB) 1817552.1
Oct. 29, 2018 (GB) 1817553.9
Oct. 29, 2018 (GB) 1817554.7
Oct. 29, 2018 (GB) 1817561.2
Oct. 29, 2018 (GB) 1817581.0

(51) Int. Cl. A24D 1/20; A24D 3/17; A24F 40/20; A24F 40/46

(52) U.S. Cl. CPC A24D 1/20; A24D 3/17; A61M 15/06; A24F 40/46; A24F 40/20

(57) ABSTRACT

The present disclosure relates to aerosol-forming articles, including an aerosol-forming article comprising an aerosol-forming substrate wherein the aerosol-forming substrate comprises a pre-formed axially-extending recess adapted to receive an external heating element. The disclosure further relates to smoking substitute articles, including HNB consumables, which may comprise a cooling element formed of, for example, plant material.

### INCORPORATION BY REFERENCE

The present disclosure is a continuation of, and claims priority to, international patent application no. PCT/EP2019/079161 filed on Oct. 25, 2019, claiming priority to application no. GB1817552.1, filed Oct. 29, 2018. The present disclosure is a continuation of, and claims priority to, international patent application no. PCT/EP2019/079181 filed on Oct. 25, 2019, claiming priority to application no. GB1817554.7, filed Oct. 29, 2018. The present disclosure is a continuation of, and claims priority to, international patent application no. PCT/EP2019/079183 filed on Oct. 25, 2019, claiming priority to application no. GB1817553.9, filed Oct. 29, 2018. The present disclosure is a continuation of, and claims priority to, international patent application no. PCT/EP2019/079197 filed on Oct. 25, 2019, claiming priority to application no. GB1817561.2, filed Oct. 29, 2018. The present disclosure is a continuation of, and claims priority to, international patent application no. PCT/EP2019/079200 filed on Oct. 25, 2019, claiming priority to application no. GB1817534.9, filed Oct. 29, 2018. The present disclosure is a continuation of, and claims priority to, international patent application no. PCT/EP2019/079208 filed on Oct. 25, 2019, claiming priority to application no. GB1817581.0, filed Oct. 29, 2018. The present disclosure is a continuation of, and claims priority to, international patent application no. PCT/EP2019/079227 filed on Oct. 25, 2019, claiming priority to application no. GB1817550.5, filed Oct. 29, 2018, and to application no. GB1817546.3, filed Oct. 29, 2018. The present disclosure is a continuation of, and claims priority to, international patent application no. PCT/EP2019/079272 filed on Oct. 25, 2019, claiming priority to application no. GB1817535.6, filed Oct. 29, 2018. The entire content of each of the applications listed above is hereby incorporated herein by reference.

### FIELD OF THE DISCLOSURE

The present disclosure relates to a consumable for use in a smoking substitute system and particularly, although not exclusively, to a heat-not-burn (HNB) consumable.

### BACKGROUND

During the manufacturing process for tobacco products, such as cigarettes, tobacco waste is produced. The tobacco waste is typically in the form of tobacco dust, leaf or stem. The tobacco waste can be collected and processed to provide what is known as reconstituted tobacco, or “recon”.

Two distinct methods are most commonly employed to manufacture reconstituted tobacco, these are i) a paper-making process (i.e. to provide “Paper Recon”), and ii) slurry casting (to provide “Slurry-type Recon”). The products that result from these distinct processes have notably different properties and characteristics. For example, slurry-type recon generally has improved aroma/flavour and nicotine content in comparison to paper recon, yet slurry-type recon is typically more easily broken and often has poorer uniformity of quality.

In slurry casting the tobacco waste is milled to a fine powder and mixed with an aqueous solvent, typically water, and combined with a binding agent. The resultant slurry may undergo further milling to reduce the particle size of the tobacco material further. The slurry is then cast on a surface and dried to form a sheet. The dried sheet may be shredded to be used within various tobacco products, for example as a cigarette filler.

In the paper-making process the tobacco waste is mechanically beaten in the presence of an aqueous solvent to digest and process the tobacco into workable fibres. Subsequently, water soluble compounds present in the tobacco are extracted into the aqueous solvent. The aqueous extract and insoluble fibrous portion are separated. The separated fibrous portion, which may also be described as a “pulp”, undergoes further processing to form a base sheet via a typical paper-making procedure. The aqueous extract is concentrated and then reapplied to the base sheet. The base sheet is then dried to form a paper.

The smoking of tobacco is generally considered to expose a smoker to potentially harmful substances. It is generally thought that a significant amount of the potentially harmful substances are generated through the heat caused by the burning and/or combustion of the tobacco and the constituents of the burnt tobacco in the tobacco smoke itself.

Conventional combustible smoking articles, such as cigarettes, typically comprise a cylindrical rod of tobacco comprising shreds of tobacco which is surrounded by a wrapper, and usually also a cylindrical filter axially aligned in an abutting relationship with the wrapped tobacco rod. The filter typically comprises a filtration material which is circumscribed by a plug wrap. The wrapped tobacco rod and the filter are joined together by a wrapped band of tipping paper that circumscribes the entire length of the filter and an adjacent portion of the wrapped tobacco rod. A conventional cigarette of this type is used by lighting the end opposite to the filter, and burning the tobacco rod. The smoker receives mainstream smoke into their mouth by drawing on the mouth end or filter end of the cigarette.

Combustion of organic material such as tobacco is known to produce tar and other potentially harmful by-products. There have been proposed various smoking substitute systems (or “substitute smoking systems”) in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapour”) that is drawn into the lungs through the mouth (inhaled) and then exhaled. The inhaled aerosol typically bears nicotine and/or flavourings without, or with fewer of, the odour and health risks associated with traditional smoking.

In general, smoking substitute systems are intended to provide a substitute for the rituals of smoking, whilst providing the user with a similar experience and satisfaction to those experienced with traditional smoking and with combustible tobacco products. Some smoking substitute systems use smoking substitute articles that are designed to resemble a traditional cigarette and are cylindrical in form with a mouthpiece at one end.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories.

There are a number of different categories of smoking substitute systems, each utilising a different smoking substitute approach.

One approach for a smoking substitute system is the so-called “heat not burn” (“HNB”) approach in which tobacco (rather than an “e-liquid”) is heated or warmed to release vapour. The tobacco may be leaf tobacco or reconstituted tobacco. The vapour may contain nicotine and/or flavourings. In the HNB approach the intention is that the tobacco is heated but not burned, i.e. the tobacco does not undergo combustion.

A typical HNB smoking substitute system may include a device and a consumable. The consumable may include the tobacco material. The device and consumable may be configured to be physically coupled together. In use, heat may be imparted to the tobacco material by a heating element of the device, wherein airflow through the tobacco material causes moisture in the tobacco material to be released as vapour. A vapour may also be formed from a carrier in the tobacco material (this carrier may for example include propylene glycol and/or vegetable glycerine) and additionally volatile compounds released from the tobacco. The released vapour may be entrained in the airflow drawn through the tobacco.

As the vapour passes through the consumable (entrained in the airflow) from an inlet to a mouthpiece (outlet), the vapour cools and condenses to form an aerosol for inhalation by the user. The aerosol will normally contain the volatile compounds.

In HNB smoking substitute systems, heating as opposed to burning the tobacco material is believed to cause fewer, or smaller quantities, of the more harmful compounds ordinarily produced during smoking. Consequently, the HNB approach may reduce the odour and/or health risks that can arise through the burning, combustion and pyrolytic degradation of tobacco.

There is a need for improved design of HNB consumables to enhance the user experience and improve the function of the HNB smoking substitute system.

The present disclosure has been devised in the light of the above considerations.

### SUMMARY OF THE DISCLOSURE

First Mode of the Disclosure: Smoking Substitute Article with Elongated Heating Element

At its most general, the first mode of the present disclosure relates to an aerosol-forming article, e.g., a smoking substitute article such as an HNB consumable an aerosol-forming substrate adapted to facilitate insertion of an elongated heating element.

According to a first aspect of the first mode, the present disclosure provides an aerosol-forming article (e.g. a smoking substitute article such as an HNB consumable) comprising an aerosol-forming substrate wherein the aerosol-forming substrate comprises a pre-formed axially-extending recess adapted to receive an external heating element.

By providing an aerosol-forming substrate having a pre-formed axially-extending recess for receiving an external heating element (i.e. formed during manufacture of the article rather than during insertion of the external heater), the user of a smoking substitute system having an external heater can easily locate the heater correctly in the aerosol-forming substrate and penetrate the aerosol-forming substrate with reduced resistance from the aerosol-forming substrate.

Optional features will now be set out. These are applicable singly or in any combination with any aspect.

As used herein, the terms “upstream” and “downstream” are intended to refer to the flow direction of the vapour/aerosol i.e. with the downstream end of the consumable being the mouth end or outlet where the aerosol exits the consumable for inhalation by the user. The upstream end of the consumable is the opposing end to the downstream end.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing.

The aerosol-forming substrate at least partly comprises plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

Preferably, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon).

The aerosol-forming substrate may comprise a gathered sheet of homogenised (e.g. paper/slurry recon) tobacco or gathered shreds/strips formed from such a sheet.

In some embodiments, the sheet used to form the aerosol-forming substrate has a grammage greater than or equal to 100 g/m², e.g. greater than or equal to 110 g/m² such as greater than or equal to 120 g/m².

The sheet may have a grammage of less than or equal to 300 g/m² e.g. less than or equal to 250 g/m² or less than or equal to 200 g/m².

The sheet may have a grammage of between 120 and 190 g/m².

The aerosol-forming substrate may comprise extruded tobacco which can produced by forming a liquid mixture of powered tobacco and a binding agent such as a gum (e.g. xanthan, guar, arabic and/or locust bean gum). The liquid mixture is heated and then extruded through a die to form the tube-shaped extrudate.

The aerosol-forming substrate may comprise at least 50 wt % plant material, e.g. at least 60 wt % plant material e.g. around 65 wt % plant material. The aerosol-forming substrate may comprise 80 wt % or less plant material e.g. 75 or 70 wt % or less plant material.

The aerosol-forming substrate may comprise one or more additives selected from humectants, flavourants, fillers, aqueous/non-aqueous solvents and binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %.

Suitable binders are known in the art and may act to bind together the components forming the aerosol-forming substrate. Binders may comprise starches and/or cellulosic binders such as methyl cellulose, ethyl cellulose, hydroxypropyl cellulose, hydroxyethyl cellulose and methyl cellulose, gums such as xanthan, guar, arabic and/or locust bean gum, organic acids and their salts such as alginic acid/sodium alginate, agar and pectins.

Preferably the binder content is 5 to 10 wt % of the aerosol-forming substrate e.g. around 6 to 8 wt %.

Suitable fillers are known in the art and may act to strengthen the aerosol-forming substrate. Fillers may comprise fibrous (non-tobacco) fillers such as cellulose fibres, lignocellulose fibres (e.g. wood fibres), jute fibres and combinations thereof.

Preferably, the filler content is 5 to 10 wt % of the aerosol-forming substrate e.g. around 6 to 9 wt %.

The aerosol-forming substrate may comprise an aqueous and/or non-aqueous solvent. In some embodiments, the aerosol forming substrate has a water content of between 5 and 10 wt % e.g. between 6-9 wt % such as between 7-9 wt %.

The flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed/dosed throughout the aerosol-forming substrate e.g. it may be added to the liquid mixture of plant material prior to extrusion.

The aerosol-forming substrate may be formed in a substantially cylindrical shape such that the consumable resembles a conventional cigarette. It may have a diameter of between 5 and 10 mm e.g. between 6 and 9 mm or 6 and 8 mm e.g. around 7 mm.

It may have an axial length of between 10 and 15 mm.

The aerosol-forming substrate may have an axial length of between 10 and 15 mm. The axially-extending recess may extend the entire axial length of the aerosol-forming substrate e.g. it may have an axial length of between 10 and 15 mm. In other embodiments, the axially-extending recess may have an axial downstream end that terminates within the aerosol-forming substrate. The axial upstream end of the axially-extending recess is at the axial upstream end of the article/consumable. The axial distance between the upstream and downstream axial ends of the axially-extending recess may be greater than 1 mm e.g. greater than 2 mm or 3 mm or 4 mm or 5 mm or 6 mm or 7 mm or 8 mm or 9 mm.

The axially-extending recess in the aerosol-forming substrate may a substantially circular radial cross-section at its upstream axial end and may have a diameter of between 1 and 3 mm e.g. around 2 mm.

The axial upstream end of the axially-extending recess may be provided at the axial centre of the upstream axial end of the aerosol-forming substrate.

The axially-extending recess may have a substantially uniform radial cross-section along its axial length (e.g. with the downstream axial end having substantially the same cross-sectional shape/area as the upstream axial end) or the axially-extending recess may taper to a reduced cross-sectional area/diameter at the downstream axial end. For example, the axially-extending recess may taper to a point.

The axially-extending recess may be formed by indenting the upstream axial end of the aerosol-forming substrate or the aerosol-forming substrate may be formed around a removable insert which leaves the shape of the recess once removed.

The aerosol-forming substrate may be circumscribed by a wrapping layer e.g. a paper wrapping layer. The wrapping layer may overlie an inner foil layer or may comprise a paper/foil laminate (with the foil innermost).

The article/consumable may comprise at least one filter element. There may be a terminal filter element at the downstream/mouth end of the article/consumable. There may be an upstream filter element upstream from the terminal filter element. The terminal and upstream filter elements may be axially adjacent or axially spaced from one another. The upstream filter element may be circumscribed by the (paper) wrapping layer.

The or at least one of the filter element(s) (e.g. the terminal filter element and/or upstream filter element) may be comprised of cellulose acetate or polypropylene tow. The or at least one of the filter element(s) (e.g. the terminal filter element and/or upstream filter element) may be comprised of activated charcoal. The or at least one of the filter element(s) (e.g. the terminal filter element and/or upstream filter element) may be comprised of paper. The or at least one of the filter element(s) (e.g. the terminal filter element and/or upstream filter element) may be comprised of extruded plant material. The or each filter element may be at least partly (e.g. entirely) circumscribed with a plug wrap e.g. a paper plug wrap.

The or each filter element may have a substantially cylindrical shape with a diameter substantially matching the diameter of the aerosol-forming substrate (with or without its associated wrapping layer). The axial length of the or each filter element may be less than 20 mm, e.g. between 8 and 15 mm, for example between 9 and 13 mm e.g. between 10 and 12 mm.

The or at least one of the filter element(s) may be a solid filter element. The or at least one of the filter element(s) may be a hollow bore filter element. The or each hollow bore filter may have a bore diameter of between 1 and 5 mm, e.g. between 2 and 4 mm or between 2 and 3 mm.

The terminal filter element (at the downstream end of the article/consumable) may be joined to the upstream elements forming the article/consumable by a circumscribing tipping layer e.g. a tipping paper layer. The tipping paper may have an axial length longer than the axial length of the terminal filter element such that the tipping paper completely circumscribes the terminal filter element plus the wrapping layer surrounding any adjacent upstream element.

The or at least one of the filter elements, e.g. the terminal filter element may include a capsule e.g. a crushable capsule (crush-ball) containing a liquid flavourant e.g. a liquid flavourant as described above. The capsule can be crushed by the user during smoking of the consumable to release the flavourant. The capsule may be located at the axial centre of the terminal filter element.

In some embodiments, the article/consumable may comprise an aerosol-cooling element which is adapted to cool the aerosol generated from the aerosol-forming substrate (by heat exchange) before being inhaled by the user.

The aerosol-cooling element will be downstream from the aerosol-forming substrate. For example, it may be between the aerosol-forming substrate and the upstream filter element and/or between the two filter elements. The aerosol cooling element may be at least partly (e.g. entirely) circumscribed by the (paper) wrapping layer.

The aerosol-cooling element may be formed of a plastics material selected from the group consisting of polylactic acid (PLA), polyvinyl chloride (PVC), polyethylene (PE) and polyethylene terephthalate (PET).

The aerosol-cooling element may be formed of a crimped/gathered sheet of material to form a structure having a high surface area with a plurality of longitudinal channels to maximise heat exchange and cooling of the aerosol.

The article/consumable may comprise a spacer element that defines a space or cavity between the aerosol-forming substrate and the downstream end of the consumable. The spacer element may comprise a cardboard tube. It may be provided between the aerosol-forming substrate and the upstream filter element and/or between the two filter elements. The spacer element may be circumscribed by the (paper) wrapping layer.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In a second aspect of the first mode of the disclosure, there is provided a system comprising an article/consumable according to the first aspect and a device comprising a heating element.

The device may be a HNB device i.e. a device adapted to heat but not combust the aerosol-forming substrate.

The device may comprise a main body for housing the heating element. The heating element may comprise an elongated e.g. rod, tube-shaped or blade heating element. The heating element may project into or surround a cavity within the main body for receiving the article/consumable.

The cross-sectional of the axially-extending recess of the aerosol-forming substrate may substantially match the cross-sectional area of the elongated heater. For example, the heater is a rod/rube heater, the diameter of the axially-extending recess may substantially match the diameter of the rod/rube heater.

The device (e.g. the main body) may further comprise an electrical power supply e.g. a (rechargeable) battery for powering the heating element. It may further comprise a control unit to control the supply of power to the heating element.

In a third aspect of the first mode of the disclosure, there is provided a method of using a system according to the second aspect, the method comprising:

inserting the article/consumable into the device such that the heating element is received in the axially-extending recess of the aerosol-forming substrate; and

heating the article/consumable using the heating element.

The skilled person will appreciate that except where mutually exclusive, a feature or parameter described in relation to any one of the above aspects of the first mode may be applied to any other aspect of the first mode. Furthermore, except where mutually exclusive, any feature or parameter described herein may be applied to any aspect of the first mode and/or combined with any other feature or parameter of the first mode described herein.

Second Mode of the Disclosure: Cooling Element Formed of Plant Material

At its most general, the second mode of the present disclosure relates to an aerosol-forming article e.g. a smoking substitute article such as an HNB consumable comprising a cooling element formed of plant material.

According to a first aspect of the second mode, the present disclosure provides an aerosol-forming article (e.g. a smoking substitute article such as an HNB consumable) comprising an aerosol-forming substrate and a downstream aerosol-cooling element axially spaced from the aerosol-forming substrate, wherein the cooling element is formed of plant material.

Known HNB consumables typically use cooling elements formed of a plastic material e.g. PLA to cool the aerosol generated from the aerosol-forming substrate (by heat exchange) before being inhaled by the user. By providing a cooling element formed of plant material, the consumable is more environmentally friendly. Furthermore, any volatile compounds in the plant material forming the cooling element may be released into the aerosol from the substrate thus increasing the concentration of volatile compounds in the aerosol and enhancing the flavour or recreational/medicinal effect obtained by the user.

Optional features will now be set out. These are applicable singly or in any combination with any aspect.

As used herein, the terms “upstream” and “downstream” are intended to refer to the flow direction of the vapour/aerosol i.e. with the downstream end of the consumable being the mouth end or outlet where the aerosol exits the consumable for inhalation by the user. The upstream end of the consumable is the opposing end to the downstream end.

The aerosol-cooling element is formed of a plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

In preferred embodiments, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

The plant material forming the cooling element may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon).

The aerosol-cooling element may be formed of a crimped/gathered sheet of plant material (e.g. paper/slurry recon tobacco) to form a structure having a high surface area with a plurality of longitudinal channels to maximise heat exchange and cooling of the aerosol.

The aerosol-cooling element may be formed of extruded plant material (e.g. extruded tobacco). For example, the aerosol-cooling material may comprise pellets, granules or chips of extruded plant material/tobacco which will create a high surface area for heat exchange.

Extruded tobacco can produced by forming a liquid mixture of powered tobacco and a binding agent such as a gum (e.g. xanthan, guar, arabic and/or locust bean gum). The liquid mixture is heated and then extruded through a die. The extrudate is dried and then may be subsequently cut into pellets, chips or granules.

The aerosol-cooling element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In contrast to the plant material cooling element, the aerosol-forming substrate is provided to be heated by a heating element to release at least one volatile compound that can form an aerosol. The aerosol-forming substrate may be located at the upstream end of the consumable. The cooling element on the other hand, will be downstream of the aerosol-forming substrate and is arranged so as not to be heated by any heating element.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

In preferred embodiments, the aerosol-forming substrate comprises tobacco which will contain nicotine as a volatile compound. Any type of tobacco may be used. This includes, but is not limited to, flue-cured tobacco, burley tobacco, Maryland tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

The aerosol-forming substrate may comprise one or more of leaf tobacco, stem tobacco, tobacco powder, tobacco dust, tobacco derivatives, expanded tobacco, homogenised tobacco, shredded tobacco, extruded tobacco, cut rag and/or reconstituted tobacco (e.g. slurry recon or paper recon).

The aerosol-forming substrate may comprise a gathered sheet of homogenised (e.g. paper/slurry recon) tobacco or gathered shreds/strips formed from such a sheet.

In some embodiments, the sheet used to form the aerosol-forming substrate has a grammage greater than or equal to 100 g/m², e.g. greater than or equal to 110 g/m² such as greater than or equal to 120 g/m².

The sheet may have a grammage of less than or equal to 300 g/m² e.g. less than or equal to 250 g/m² or less than or equal to 200 g/m².

The sheet may have a grammage of between 120 and 190 g/m².

The aerosol-forming substrate may comprise at least 50 wt % plant material, e.g. at least 60 wt % plant material e.g. around 65 wt % plant material. The aerosol-forming substrate may comprise 80 wt % or less plant material e.g. 75 or 70 wt % or less plant material.

The aerosol-forming substrate may comprise the same or different plant material e.g. the same or different type/form of tobacco as the tobacco cooling element.

The aerosol-forming substrate may comprise one or more additives selected from humectants, flavourants, fillers, aqueous/non-aqueous solvents and binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %. Suitable binders are known in the art and may act to bind together the components forming the aerosol-forming substrate. Binders may comprise starches and/or cellulosic binders such as methyl cellulose, ethyl cellulose, hydroxypropyl cellulose, hydroxyethyl cellulose and methyl cellulose, gums such as xanthan, guar, arabic and/or locust bean gum, organic acids and their salts such as alginic acid/sodium alginate, agar and pectins.

Preferably the binder content is 5 to 10 wt % of the aerosol-forming substrate e.g. around 6 to 8 wt %.

Suitable fillers are known in the art and may act to strengthen the aerosol-forming substrate. Fillers may comprise fibrous (non-tobacco) fillers such as cellulose fibres, lignocellulose fibres (e.g. wood fibres), jute fibres and combinations thereof.

Preferably, the filler content is 5 to 10 wt % of the aerosol-forming substrate e.g. around 6 to 9 wt %.

The aerosol-forming substrate may comprise an aqueous and/or non-aqueous solvent. In some embodiments, the aerosol forming substrate has a water content of between 5 and 10 wt % e.g. between 6-9 wt % such as between 7-9 wt %.

The flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed/dosed throughout the first and/or second aerosol-forming material.

The aerosol-forming substrate may be formed in a substantially cylindrical shape such that the consumable resembles a conventional cigarette. It may have a diameter of between 5 and 10 mm e.g. between 6 and 9 mm or 6 and 8 mm e.g. around 7 mm.

The aerosol-forming substrate may be circumscribed by a wrapping layer e.g. a paper wrapping layer. The wrapping layer may overlie an inner foil layer or may comprise a paper/foil laminate (with the foil innermost).

The aerosol-cooling element is downstream and axially spaced from the aerosol-forming substrate. It may be axially spaced from the aerosol-forming substrate by an upstream filter element. The upstream filter element may be axially adjacent the downstream end of the aerosol-forming substrate or may be axially spaced from the aerosol-forming substrate.

There may a downstream filter element axially downstream (e.g. axially adjacent) the aerosol-cooling element. In some embodiments, the aerosol-cooling element is sandwiched between the upstream and downstream filter elements. The downstream filter element may be a terminal filter element i.e. at the axial downstream end of the article/consumable.

The upstream and/or downstream filter(s) may be comprised of cellulose acetate or polypropylene tow. The upstream and/or downstream filter(s) may be comprised of activated charcoal. The upstream and/or downstream filter(s) may be comprised of paper. The upstream and/or downstream filter(s) may be at least partly (e.g. entirely) circumscribed with a plug wrap e.g. a paper plug wrap. The upstream filter element may be at least partly (e.g. fully) circumscribed by the (paper) wrapping layer.

The upstream and/or downstream filter(s) may have a substantially cylindrical shape with a diameter substantially matching the diameter of the aerosol-forming substrate (with or without its associated wrapping layer). The axial length of the upstream and/or downstream filter(s) may be less than 20 mm, e.g. between 8 and 15 mm, for example between 9 and 13 mm e.g. between 10 and 12 mm.

The upstream and/or downstream filter(s) may be a solid filter element. The upstream and/or downstream filter(s) may be a hollow bore filter element. The or each hollow bore filter may have a bore diameter of between 1 and 5 mm, e.g. between 2 and 4 mm or between 2 and 3 mm.

The terminal filter element (at the downstream end of the article/consumable) may be joined to the upstream elements forming the article/consumable (e.g. to the aerosol-cooling element) by a circumscribing tipping layer e.g. a tipping paper layer. The tipping paper may have an axial length longer than the axial length of the terminal filter element such that the tipping paper completely circumscribes the terminal filter element plus the wrapping layer surrounding any adjacent upstream element (e.g. the aerosol-cooling element).

The upstream and/or downstream filter(s) may include a capsule e.g. a crushable capsule (crush-ball) containing a liquid flavourant e.g. a liquid flavourant as described above. The capsule can be crushed by the user during smoking of the consumable to release the flavourant. The capsule may be located at the axial centre of the terminal filter element.

The article/consumable may comprise a spacer element that defines a space or cavity between the aerosol-forming substrate and the downstream end of the consumable. The spacer element may comprise a cardboard tube. The spacer element may be circumscribed by the (paper) wrapping layer.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In a second aspect of the second mode of the present disclosure, there is provided a system comprising an article/consumable according to the first aspect and a device comprising a heating element.

The device may be a HNB device i.e. a device adapted to heat but not combust the aerosol-forming substrate. The device will not directly heat the plant material cooling element.

The device may comprise a main body for housing the heating element. The heating element may comprise an elongated e.g. rod, tube-shaped or blade heating element. The heating element may project into or surround a cavity within the main body for receiving the article/consumable.

The device (e.g. the main body) may further comprise an electrical power supply e.g. a (rechargeable) battery for powering the heating element. It may further comprise a control unit to control the supply of power to the heating element.

In a third aspect of the second mode of the present disclosure, there is provided a method of using a system according to the second aspect, the method comprising: inserting the article/consumable into the device; and heating the article/consumable using the heating element.

In some embodiments, the method comprises inserting the article/consumable into a cavity within the main body and penetrating the article/consumable with the heating element upon insertion of the article/consumable. For example, the heating element may penetrate the aerosol-forming substrate in the article/consumable. The plant material cooling element will not be penetrated and/or heated directly by the heating element. For example, the cooling element may remain external to the device when the consumable/article is inserted.

The skilled person will appreciate that except where mutually exclusive, a feature or parameter described in relation to any one of the above aspects of the second mode may be applied to any other aspect of the second mode. Furthermore, except where mutually exclusive, any feature or parameter described herein may be applied to any aspect of the second mode and/or combined with any other feature or parameter of the second mode described herein.

Third Mode: Extruded Plant Material

At its most general, the third mode of the present disclosure relates to an aerosol-forming article e.g. a smoking substitute article such as an HNB consumable an aerosol-forming substrate at least partly formed of an extruded plant material.

According to a first aspect of the third mode, the present disclosure provides an aerosol-forming article (e.g. a smoking substitute article such as an HNB consumable) comprising an aerosol-forming substrate wherein the aerosol-forming substrate at least partly comprises a rod of extruded plant material having an axial bore adapted to receive an external heating element.

Extruded plant material is typically more compacted/more dense than other types of plant material typically used in smoking substitute articles. By providing an aerosol-forming substrate formed of or comprising a rod of extruded plant material having an empty axial bore for receiving an external heating element, the user of a smoking substitute system having an external heater is provided with an aerosol having an increased concentration of volatile compounds and thus an enhanced medicinal/recreational effect.

Optional features will now be set out. These are applicable singly or in any combination with any aspect of the third mode.

As used herein, the terms “upstream” and “downstream” are intended to refer to the flow direction of the vapour/aerosol i.e. with the downstream end of the consumable being the mouth end or outlet where the aerosol exits the consumable for inhalation by the user. The upstream end of the consumable is the opposing end to the downstream end.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing.

The aerosol-forming substrate at least partly comprises extruded plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

Preferably, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

Extruded tobacco can be produced by forming a liquid mixture of powered tobacco and a binding agent such as a gum (e.g. xanthan, guar, arabic and/or locust bean gum). The liquid mixture is heated and then extruded through a die to form the tube-shaped extrudate.

The aerosol-forming substrate may comprise at least 50 wt % plant material, e.g. at least 60 wt % plant material e.g. around 65 wt % plant material. The aerosol-forming substrate may comprise 80 wt % or less plant material e.g. 75 or 70 wt % or less plant material.

The aerosol-forming substrate may comprise one or more additives selected from humectants, flavourants, fillers, aqueous/non-aqueous solvents and binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %

Suitable binders are known in the art and may act to bind together the components forming the aerosol-forming substrate. Binders may comprise starches and/or cellulosic binders such as methyl cellulose, ethyl cellulose, hydroxypropyl cellulose, hydroxyethyl cellulose and methyl cellulose, gums such as xanthan, guar, arabic and/or locust bean gum, organic acids and their salts such as alginic acid/sodium alginate, agar and pectins.

Preferably the binder content is 5 to 10 wt % of the aerosol-forming substrate e.g. around 6 to 8 wt %.

Suitable fillers are known in the art and may act to strengthen the aerosol-forming substrate. Fillers may comprise fibrous (non-tobacco) fillers such as cellulose fibres, lignocellulose fibres (e.g. wood fibres), jute fibres and combinations thereof.

Preferably, the filler content is 5 to 10 wt % of the aerosol-forming substrate e.g. around 6 to 9 wt %.

The aerosol-forming substrate may comprise an aqueous and/or non-aqueous solvent. In some embodiments, the aerosol forming substrate has a water content of between 5 and 10 wt % e.g. between 6-9 wt % such as between 7-9 wt %.

The flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed/dosed throughout the aerosol-forming substrate e.g. it may be added to the liquid mixture of plant material prior to extrusion.

The aerosol-forming substrate may be formed in a substantially cylindrical shape such that the consumable resembles a conventional cigarette. It may have a diameter of between 5 and 10 mm e.g. between 6 and 9 mm or 6 and 8 mm e.g. around 7 mm.

It may have an axial length of between 10 and 15 mm.

The extruded rod of plant material may form the entire aerosol-forming substrate in which case it may have an axial length of between 10 and 15 mm. The axial bore through the extruded rod of plant material may extend through the entire axial length of the extrudate e.g. it may have an axial length of between 10 and 15 mm. The axial upstream end of the axial bore is at the axial upstream end of the article/consumable.

The aerosol-forming substrate may be circumscribed by a wrapping layer e.g. a paper wrapping layer. The wrapping layer may overlie an inner foil layer or may comprise a paper/foil laminate (with the foil innermost).

The axial bore in the extruded rod of plant material may have a diameter of between 1 and 3 mm e.g. around 2 mm.

The article/consumable may comprise at least one filter element. There may be a terminal filter element at the downstream/mouth end of the article/consumable. There may be an upstream filter element upstream of the axial downstream end. The upstream filter element may be circumscribed by the (paper) wrapping layer. The terminal and upstream filter elements may be axially adjacent or may be axially spaced.

The or at least one of the filter element(s) (e.g. the terminal filter element and/or upstream filter element) may be comprised of cellulose acetate or polypropylene tow. The or at least one of the filter element(s) (e.g. the terminal filter element and/or upstream filter element) may be comprised of activated charcoal. The or at least one of the filter element(s) (e.g. the terminal filter element and/or upstream filter element) may be comprised of paper. The or at least one of the filter element(s) (e.g. the terminal filter element and/or upstream filter element) may be comprised of plant material e.g. extruded plant material. The or each filter element may be at least partly (e.g. entirely) circumscribed with a plug wrap e.g. a paper plug wrap.

The or each filter element may have a substantially cylindrical shape with a diameter substantially matching the diameter of the aerosol-forming substrate (with or without its associated wrapping layer). The axial length of the or each filter element may be less than 20 mm, e.g. between 8 and 15 mm, for example between 9 and 13 mm e.g. between 10 and 12 mm.

The or at least one of the filter element(s) may be a solid filter element. The or at least one of the filter element(s) may be a hollow bore filter element. The or each hollow bore filter may have a bore diameter of between 1 and 5 mm, e.g. between 2 and 4 mm or between 2 and 3 mm.

The terminal filter element (at the downstream end of the article/consumable) may be joined to the upstream elements forming the article/consumable by a circumscribing tipping layer e.g. a tipping paper layer. The tipping paper may have an axial length longer than the axial length of the terminal filter element such that the tipping paper completely circumscribes the terminal filter element plus the wrapping layer surrounding any adjacent upstream element.

The or at least one of the filter elements e.g. the terminal filter element may include a capsule e.g. a crushable capsule (crush-ball) containing a liquid flavourant e.g. a liquid flavourant as described above.

The capsule can be crushed by the user during smoking of the consumable to release the flavourant. The capsule may be located at the axial centre of the terminal filter element.

In some embodiments, the article/consumable may comprise an aerosol-cooling element which is adapted to cool the aerosol generated from the aerosol-forming substrate (by heat exchange) before being inhaled by the user.

The aerosol-cooling element will be downstream from the aerosol-forming substrate. For example, it may be between the aerosol-forming substrate and a/the filter element and/or between two filter elements. The aerosol cooling element may be at least partly (e.g. entirely) circumscribed by the (paper) wrapping layer.

The aerosol-cooling element may be formed of a plastics material selected from the group consisting of polylactic acid (PLA), polyvinyl chloride (PVC), polyethylene (PE) and polyethylene terephthalate (PET). The aerosol-cooling element may be formed of a crimped/gathered sheet of material to form a structure having a high surface area with a plurality of longitudinal channels to maximise heat exchange and cooling of the aerosol.

The article/consumable may comprise a spacer element that defines a space or cavity between the aerosol-forming substrate and the downstream end of the consumable. It may be provided between the aerosol-forming substrate and a/the filter element and/or between two filter elements. The spacer element may comprise a tubular element e.g. cardboard or plastic material tube. The spacer element may be circumscribed by the (paper) wrapping layer.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In a second aspect of the third mode, there is provided a system comprising an article/consumable according to the first aspect and a device comprising a heating element.

The device may be a HNB device i.e. a device adapted to heat but not combust the aerosol-forming substrate.

The device may comprise a main body for housing the heating element. The heating element may comprise an elongated e.g. rod, tube-shaped or blade heating element. The heating element may project into or surround a cavity within the main body for receiving the article/consumable.

The diameter of the axial bore of the extruded rod of tobacco preferably matches the diameter of the elongated rod/tube heater.

The device (e.g. the main body) may further comprise an electrical power supply e.g. a (rechargeable) battery for powering the heating element. It may further comprise a control unit to control the supply of power to the heating element.

In a third aspect of the third mode, there is provided a method of using a system according to the second aspect, the method comprising: inserting the article/consumable into the device such that the heating element is received in the axial bore of the aerosol-forming substrate; and heating the article/consumable using the heating element.

The skilled person will appreciate that except where mutually exclusive, a feature or parameter described in relation to any one of the above aspects of the third mode may be applied to any other aspect of the third mode. Furthermore, except where mutually exclusive, any feature or parameter described herein may be applied to any aspect of the third mode and/or combined with any other feature or parameter of the third mode described herein.

Fourth Mode: Filter Having One or More Bores

At its most general, the fourth mode of the present disclosure relates to an aerosol-forming article e.g. a smoking substitute article such as an HNB consumable comprising a filter having one or more bores.

According to a first aspect of the fourth mode, there is provided a smoking substitute system comprising: a device comprising an elongate heating element; and an aerosol-forming article, the article comprising an aerosol-forming substrate having a longitudinal axis along which the heating element is received in use, and a filter element axially downstream of, and adjacent to, the substrate, the filter element comprising at least one bore having an opening at an upstream axial end of the filter element, the or each opening being arranged so as to be non-coincident with the longitudinal axis of the substrate.

When the heater is inserted into the aerosol-forming substrate, the material that forms the aerosol-forming substrate (e.g. plant material such as tobacco) may be displaced by the heating element. By arranging the openings in the filter element so as to be non-coincident with the longitudinal axis of the substrate (along which the heater is received), the openings (and corresponding bores) may be less susceptible to blockages caused by displaced substrate material entering the openings.

The term non-coincident means that the openings do not intersect with, or are not aligned with, the longitudinal axis. That is, the openings are spaced (in a transverse direction) from the longitudinal axis. The longitudinal axis of the substrate may be substantially coaxial with a central (longitudinal) axis of the aerosol-forming article.

As used herein, the terms “upstream” and “downstream” are intended to refer to the flow direction of the vapour/aerosol i.e. with the downstream end of the article being the mouth end or outlet where the aerosol exits the article for inhalation by the user. The upstream end of the article is the opposing end to the downstream end.

Optional features will now be set out. These are applicable singly or in any combination with any aspect.

In some embodiments the upstream axial end of the filter element comprises a projected region defined by a projection (i.e. projected along the longitudinal axis) of the transverse profile of the heating element, when received in the substrate, onto the upstream axial end of the filter element. In general, when the heating element is received in substrate, a downstream end of the heating element (i.e. distal from its mounting to the device) may be longitudinally spaced from the upstream axial end of the filter element. However, if (in a hypothetical scenario) the heating element were to be moved along the longitudinal axis so as to extend through the upstream axial end of the filter element, the projected region would be equivalent to a region of the upstream axial end that intersects with the transverse profile of the heating element. For example, in embodiments where the heating element has a circular transverse profile aligned centrally on the longitudinally axis, the projected region would be a corresponding circular region on the upstream axial end of the filter element having the same diameter as the heating element and aligned centrally on the longitudinal axis.

In some embodiments, the or each opening may be located so as to be at least partially outside of the projected region. For example, the or each opening may have a portion of e.g. at least 30% of its area outside of the region, or e.g. at least 50% of its area outside of the projected region, or at least 80% of its area outside of the projected region. The or each opening may be arranged so as to be fully outside of the projected region.

In some embodiments, the or each opening may be arranged so as to be closer to a perimeter of the projected region than a periphery (i.e. a peripheral edge) of the upstream axial end of the filter element. In some cases, locating openings too close to a peripheral edge of the filter element could be detrimental to the structural integrity of the filter element.

In some embodiments, the or each opening may be arranged such that a perimeter of the or each opening is substantially contiguous with a perimeter of the projected region (i.e. the openings may be directly adjacent the projected region). In other words, the or each opening may be fully outside, but not spaced from, the projected region. Alternatively, the or each opening may be spaced from the projected region.

In some embodiment, the filter element may comprise a plurality of bores (e.g. two, three, four etc. bores), each comprising a respective opening at the upstream axial end of the filter element (i.e. the filter element may comprise a plurality of openings). The plurality of openings may be spaced (e.g. evenly) about the perimeter of the projected region. The plurality of bores may be arranged in a circle about the longitudinal axis. A plurality of bores may help to create a pressure drop in fluid flowing across the filter element, which may be desirable with respect to user experience.

The or each bore may extend so as to be substantially parallel to the longitudinal axis. Alternatively, the or each bore extend at an angle relative to a longitudinal axis, or may e.g. have a curved (rather than linear) path.

In some embodiments, the heating element may have a substantially circular transverse profile. In this case, and as is set forth above, the projected region would be substantially circular. The heating element may alternatively have a rectangular transverse profile (e.g. where the heating element is in the form of a blade heating element). As above, the corresponding projected region of the heating element would be rectangular. In other embodiments the heating element may have an elliptical, triangular, hexagonal, etc. transverse profile. The heating element may comprise an elongated e.g. rod, tube-shaped or blade heating element.

When circular, the diameter of the heating element may be between 1 mm and 3 mm. The diameter of the heating element may be between 1.5 mm and 2.5 mm. The diameter of the heating element may be around 2 mm. Consequently, the diameter of the projected region may be between 1 mm and 3 mm, or between 1.5 mm and 2.5 mm, or around 2 mm.

The or each bore may extend partway or fully through the filter element (e.g. in a longitudinal direction). The or each opening may be circular (or may e.g. be square, rectangular, triangular, etc.). When circular, the or each opening may have a diameter of between 0.5 mm and 1.5 mm. The or each opening may have a diameter of around 1 mm.

In some embodiments the or each opening may be arranged on the upstream axial end of the filter element such that a centre of the or each opening is spaced from the longitudinal axis of the filter element by a distance that is between 1 mm and 3 mm or e.g. between 1 mm and 2 mm, or e.g. around 1.5 mm. The centre of each opening may spaced from a central longitudinal axis of the filter element by a distance that is greater than 1.5 mm (e.g. where each opening has a diameter of 1 mm and the heating element has a diameter of 2 mm).

In some embodiments the device comprises a main body for housing the heating element and a cavity for receipt of the aerosol-forming article. The cavity may be arranged such that, when the aerosol-forming article is received in the cavity, the heating element is received in (e.g. inserted into) the aerosol-forming substrate along the longitudinal axis. The device may comprise guide surfaces so as to align the aerosol-forming substrate with the heating element (i.e. such that the heating element is aligned along the longitudinal axis). In this way, the position of the heating element may be consistent across aerosol-forming articles of the same type (e.g. same dimensions, shape, etc.)

The device may be a heat-not-burn (HNB) device i.e. a device adapted to heat but not combust the aerosol-forming substrate. The device (e.g. the main body) may further comprise an electrical power supply e.g. a (rechargeable) battery for powering the heating element. It may further comprise a control unit to control the supply of power to the heating element.

The aerosol-forming article is preferably a HNB consumable.

The aerosol-forming substrate of the article/consumable may be capable of being heated to release at least one volatile compound that can form an aerosol. The aerosol-forming substrate may be located at the upstream end of the article/consumable.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

Preferably, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon).

The aerosol-forming substrate may comprise wt % plant material.

The aerosol-forming substrate may comprise one or more additives selected from humectants, flavourants, fillers and binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %.

Suitable binders are known in the art and may comprise starches and/or cellulosic binders such as methyl cellulose, ethyl cellulose, hydroxypropyl cellulose, hydroxyethyl cellulose and methyl cellulose, gums such as xanthan, guar, arabic and/or locust bean gum, organic acids and their salts such as alginic acid/sodium alginate, agar and pectins.

Preferably the binder content is 5 to 10 wt % of the aerosol-forming substrate e.g. around 6 to 8 wt %.

Suitable fillers are known in the art and may comprise fibrous fillers such as cellulose fibres.

Preferably, the filler content is 5 to 10 wt % of the aerosol-forming substrate e.g. around 6 to 9 wt %.

The flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g. between 6 and 9 mm or 6 and 8 mm e.g. around 7 mm. It may have an axial length of between 10 and 15 mm e.g. between 11 and 14 mm such as around 12 or 13 mm.

The aerosol-forming substrate may be circumscribed by a wrapping layer e.g. a paper wrapping layer. The wrapping layer may overlie an inner foil layer or may comprise a paper/foil laminate (with the foil innermost).

In some embodiments, the aerosol-forming article may comprise a further filter element in the form of a terminal hollow bore filter element located at a downstream (i.e. mouth) end of the aerosol-forming article/consumable. So as to differentiate from the terminal filter element, the filter element described above (having a plurality of bores) will hereafter be referred to as the ‘upstream’ filter element.

The or at least one of the filter element(s) (e.g. the upstream filter element and/or terminal filter element) may be comprised of cellulose acetate or polypropylene tow. The at least one filter element (e.g. the upstream filter element and/or terminal filter element) may be comprised of activated charcoal. The terminal filter element may be comprised of paper. The or each filter element may be circumscribed with a plug wrap e.g. a paper plug wrap.

The or each filter element may have a substantially cylindrical shape with a diameter substantially matching the diameter of the aerosol-forming substrate (with or without its associated wrapping layer). The axial length of the or each filter element may be less than 20 mm, e.g. between 8 and 15 mm, for example between 9 and 13 mm e.g. between 10 and 12 mm.

The terminal filter element may be a solid filter element. Alternatively, the terminal filter element may be a hollow bore filter element. The terminal filter element may have a bore diameter of between 1 and 5 mm, e.g. between 2 and 4 mm or between 2 and 3 mm.

There may be more than two filter elements which may be adjacent one another or which may be spaced apart. The upstream filter element, and/or any additional filter element(s) upstream of the terminal filter element, may be at least partly (e.g. entirely) circumscribed by the (paper) wrapping layer.

Additional filter element(s) upstream of the terminal filter element may be joined to the terminal filter element by a circumscribing tipping layer e.g. a tipping paper layer. The tipping paper may have an axial length longer than the axial length of the terminal filter element such that the tipping paper completely circumscribes the terminal filter element plus the wrapping layer surrounding any adjacent filter element (i.e. upstream of the terminal filter element).

The or at least one of the filter elements e.g. the terminal filter element may include a capsule e.g. a crushable capsule (crush-ball) containing a liquid flavourant e.g. any of the flavourants listed above. The capsule can be crushed by the user during smoking of the article/consumable to release the flavourant. The capsule may be located at the axial centre of the terminal filter element.

In some embodiments, the article/consumable may comprise an aerosol-cooling element which is adapted to cool the aerosol generated from the aerosol-forming substrate (by heat exchange) before being inhaled by the user.

The aerosol-cooling element will be downstream from the aerosol-forming substrate. For example, it may be between the upstream filter element and the terminal filter. The aerosol cooling element may be at least partly (e.g. completely) circumscribed by the (paper) wrapping layer.

The aerosol-cooling element may be formed of a plastics material selected from the group consisting of polylactic acid (PLA), polyvinyl chloride (PVC), polyethylene (PE) and polyethylene terephthalate (PET). The aerosol-cooling element may be formed of a crimped/gathered sheet of material to form a structure having a high surface area with a plurality of longitudinal bores to maximise heat exchange and cooling of the aerosol.

The article/consumable may comprise a spacer element that defines a space or cavity or chamber between the aerosol-forming substrate and the downstream end of the article/consumable. The spacer may be located between the upstream filter element and the terminal filter. The spacer acts to allow both cooling and mixing of the aerosol. The spacer element may comprise a cardboard tube. The spacer element may be at least partly (e.g. entirely) circumscribed by the (paper) wrapping layer.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In a second aspect of the fourth mode, there is provided an aerosol-forming article comprising an aerosol-forming substrate having a longitudinal axis along which a heater may be received in use, and a filter element axially downstream of, and adjacent to, the substrate, the filter element comprising at least one bore having an opening at an upstream axial end of the filter element, the or each bore being arranged so as to be non-coincident with the longitudinal axis of the aerosol-forming substrate.

In some embodiments the longitudinal axis may be centrally located with respect to the aerosol-forming substrate.

In some embodiment, the filter element may comprise a plurality of bores (e.g. two, three, four etc. bores), each comprising a corresponding opening at the upstream axial end of the filter element (i.e. the filter element may comprise a plurality of openings. The plurality of openings at the upstream axial end may be arranged in a circle about the longitudinal axis. Alternatively, the plurality of openings may be arranged in a rectangle about the longitudinal axis.

In some embodiments, the centre of each opening (in the upstream side of the filter element) may be spaced from the longitudinal axis by a distance of between 1 mm and 3 mm or e.g. between 1 mm and 2 mm, or e.g. around 1.5 mm. The centre of each opening may be spaced from the longitudinal axis by a distance that is greater than 1.5 mm (e.g. where each opening has a diameter of 1 mm and the heating element has a diameter of 2 mm).

The or each bore may extend partway or fully through the filter element (e.g. in a longitudinal direction). The or each opening may be circular (or may e.g. be square, rectangular, triangular, etc.). When circular, the or each opening may have a diameter of between 0.5 mm and 1.5 mm. The or each opening may have a diameter of around 1 mm.

In some embodiments the filter element may be in the form of an upstream filter element, and the aerosol-forming article may comprise a further filter element in the form of a terminal hollow bore filter element located at a downstream end of the article (i.e. downstream of the substrate and the upstream filter element).

In some embodiments the aerosol-forming substrate may further comprise a spacer element located between the terminal hollow bore filter element and the upstream filter element.

In some embodiments the upstream axial end of the filter element may comprise a projected region defined by a projection (i.e. projected along the longitudinal axis) of the transverse profile of a heating element, when received in the substrate (i.e. in use, when the article is used in e.g. smoking substitute device), onto the upstream axial end of the filter element. For example, in embodiments where the article is for use with a heating element having a circular transverse profile aligned centrally on the longitudinally axis, the projected region would be a corresponding circular region on the upstream axial end of the filter element having the same diameter as the heating element and aligned centrally on the longitudinal axis.

In some embodiments, the or each opening may be located so as to be at least partially outside of the projected region. For example, the or each opening may have a portion of e.g. at least 30% of its area outside of the region, or e.g. at least 50% of its area outside of the projected region, or at least 80% of its area outside of the projected region. The or each opening may be arranged so as to be fully outside of the projected region.

In some embodiments, the or each opening may be arranged so as to be closer to a perimeter of the projected region than a periphery (i.e. a peripheral edge) of the upstream axial end of the filter element. In some cases, locating openings too close to a peripheral edge of the filter element could be detrimental to the structural integrity of the filter element.

In some embodiments, the or each opening may be arranged such that a perimeter of the or each opening is substantially contiguous with a perimeter of the projected region (i.e. the openings may be directly adjacent the projected region). In other words, the or each opening may be fully outside, but not spaced from, the projected region. Alternatively, the or each opening may be spaced from the projected region.

In some embodiments, the article may be for use with a heating element having a substantially circular transverse profile. In this case, and as is set forth above, the projected region would be substantially circular. The article may alternatively be for use with a heating element having a rectangular transverse profile (e.g. where the heating element is in the form of a blade heating element). As above, the corresponding projected region would be elliptical.

When circular, the diameter of the projected region may be between 1 mm and 3 mm. The diameter of the projected region may be between 1.5 mm and 2.5 mm. The diameter of the projected region may be around 2 mm.

The aerosol-forming article of the second aspect may be as otherwise described with respect to the first aspect.

In a third aspect of the fourth mode, there is provided a method of using a smoking substitute system according to the second aspect, the method comprising inserting the article/consumable into the device, and heating the article/consumable using the heating element.

In some embodiments, the method may comprise inserting the article/consumable into a cavity within the main body and, upon insertion of the article/consumable, penetrating the article/consumable with the heating element along the longitudinal axis of the aerosol-forming substrate.

The skilled person will appreciate that except where mutually exclusive, a feature or parameter described in relation to any one of the above aspects of the fourth mode may be applied to any other aspect of the fourth mode. Furthermore, except where mutually exclusive, any feature or parameter described herein may be applied to any aspect of the fourth mode and/or combined with any other feature or parameter of the fourth mode described herein.

Fifth Mode of the Disclosure: Disposable in a More Environmentally Friendly Manner

At its most general, the fifth mode of the present disclosure relates to an aerosol-forming article e.g. a smoking substitute article such as an HNB consumable which is more easily disposable, for example disposable in a more environmentally-friendly manner. The aerosol-forming article comprises a means to divide the consumable to provide more easily disposable individual components. The disclosure also provides an improved package for housing aerosol-forming articles before and after their use.

According to a first aspect of the fifth mode, there is provided an aerosol-forming article (e.g. a smoking substitute article such as an HNB consumable) comprising a first section and a second section, the first section comprising an aerosol-forming substrate adapted to be heated by an external heater, wherein the first section is separable from the second section.

In this way, an aerosol-forming article is provided which offers a wider range of disposal options. The first and second sections are separable such that, after use, the article may be split into separate first and second sections. Since the first section contains an aerosol-forming substrate, after separation, the aerosol-forming substrate (in the first section) may be disposed of separately from other components (in the second section). The second section or a portion thereof may for example be disposed of with home waste or recycled without contamination by the aerosol-forming substrate (e.g. tobacco) present in the first section. The first section may be disposed of separately, for example into an ashtray or by composting. The article therefore offers a more versatile alternative to articles which cannot be easily separated into multiple sections and have hitherto been disposed of as a single unit regardless of the nature of the different component parts.

Optional features will now be set out. These are applicable singly or in any combination with any aspect. The aerosol-forming article is preferably a heat-not-burn (HNB) consumable.

The aerosol-forming article comprises an aerosol-forming substrate which is within the first section of the article. The aerosol-forming substrate is capable of being heated to release at least one volatile compound that can form an aerosol. The aerosol-forming substrate may be located at the upstream end of the article/consumable.

As used herein, the terms “upstream” and “downstream” are intended to refer to the flow direction of the vapour/aerosol i.e. with the downstream end of the article/consumable being the mouth end or outlet where the aerosol exits the article/consumable for inhalation by the user. The upstream end of the article/consumable is the opposing end to the downstream end.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

Preferably, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon).

The aerosol-forming substrate may comprise a gathered sheet of homogenised (e.g. paper/slurry recon) tobacco or gathered shreds/strips formed from such a sheet.

In some embodiments, the sheet used to form the aerosol-forming substrate has a grammage greater than or equal to 100 g/m², e.g. greater than or equal to 110 g/m² such as greater than or equal to 120 g/m².

The sheet may have a grammage of less than or equal to 300 g/m² e.g. less than or equal to 250 g/m² or less than or equal to 200 g/m².

The sheet may have a grammage of between 120 and 190 g/m².

The aerosol-forming substrate may comprise at least 50 wt % plant material, e.g. at least 60 wt % plant material e.g. around 65 wt % plant material. The aerosol-forming substrate may comprise 80 wt % or less plant material e.g. 75 or 70 wt % or less plant material. The aerosol-forming substrate may comprise from 50 to 80 wt % plant material, for example from 50 to 75 wt %, from 55 to 80 wt %, from 55 to 75 wt %, from 50 to 70 wt %, from 55 to 70 wt %, from 60 to 75 wt % or from 60 to 70 wt %.

The aerosol-forming substrate may comprise one or more additives selected from humectants, flavourants, fillers, aqueous/non-aqueous solvents and binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %

Suitable binders are known in the art and may act to bind together the components forming the aerosol-forming substrate. Binders may comprise starches and/or cellulosic binders such as methyl cellulose, ethyl cellulose, hydroxypropyl cellulose, hydroxyethyl cellulose and methyl cellulose, gums such as xanthan, guar, arabic and/or locust bean gum, organic acids and their salts such as alginic acid/sodium alginate, agar and pectins.

Preferably the binder content is 5 to 10 wt % of the aerosol-forming substrate e.g. around 6 to 8 wt %.

Suitable fillers are known in the art and may act to strengthen the aerosol-forming substrate. Fillers may comprise fibrous (non-tobacco) fillers such as cellulose fibres, lignocellulose fibres (e.g. wood fibres), jute fibres and combinations thereof.

Preferably, the filler content is 5 to 10 wt % of the aerosol-forming substrate e.g. around 6 to 9 wt %.

The aerosol-forming substrate may comprise an aqueous and/or non-aqueous solvent. In some embodiments, the aerosol forming substrate has a water content of between 5 and 10 wt % e.g. between 6-9 wt % such as between 7-9 wt %.

The flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g. between 6 and 9 mm or 6 and 8 mm e.g. around 7 mm. It may have an axial length of between 10 and 15 mm e.g. between 11 and 14 mm such as around 12 or 13 mm.

The aerosol-forming substrate may be circumscribed by a wrapping layer e.g. a paper wrapping layer. Thus the first section may comprise solely the aerosol-forming substrate and the wrapping layer. For example, the first section may be devoid of any integral heating element.

In some embodiments the first section consists of biodegradable constituents. In other words, in some embodiments the first section contains only biodegradable components. Herein, the term “biodegradable” takes its usual meaning, indicating a material which is capable of being degraded or decomposed by bacteria, fungi or other living organisms. In some embodiments, the term “biodegradable” indicates a material which is at least 30 wt % degraded after 46 days as determined according to the ISO 11734 standard of 1995 (anaerobic biodegradability in municipal wastewater treatment plant sludge at 35° C.), for example at least 35 wt % degraded, at least 40 wt % degraded, at least 45 wt % degraded or at least 50 wt % degraded. Cellulose acetate fibers of the type used in cellulose acetate tow filters (DS=2.45) are 27 wt % degraded after 46 days as determined according to the ISO 11734 standard (Gartiser et al., “Assessment of Several Test Methods for the Determination of the Anaerobic Biodegradability of Polymers”, J. Polymer Environ., Vol. 6, No. 3, 1998, pp. 159-173). In some embodiments the first section consists of biodegradable constituents which comprise the aerosol-forming substrate and an outer wrapping layer.

The article/consumable may comprise at least one filter element in the second section. There may be a terminal filter element in the second section at the downstream/mouth end of the article/consumable. There may also be an upstream filter element upstream from the downstream axial end. The terminal and upstream filter elements may be axially adjacent or axially spaced from one another.

The or at least one of the filter element(s) (e.g. the terminal filter element and/or the upstream filter element) may be comprised of cellulose acetate or polypropylene tow. The or at least one of the filter element(s) (e.g. the terminal filter element and/or the upstream filter element) may be comprised of activated charcoal. The or at least one of the filter element(s) (e.g. the terminal filter element and/or the upstream filter element) may be comprised of paper. The or each filter element may be circumscribed with a plug wrap e.g. a paper plug wrap.

The or each filter element may have a substantially cylindrical shape with a diameter substantially matching the diameter of the aerosol-forming substrate (with or without its associated wrapping layer). The axial length of the or each filter element may be less than 20 mm, e.g. between 8 and 15 mm, for example between 9 and 13 mm e.g. between 10 and 12 mm.

The or at least one of the filter element(s) may be a solid filter element. The or at least one of the filter element(s) may be a hollow bore filter element. The or each hollow bore filter may have a bore diameter of between 1 and 5 mm, e.g. between 2 and 4 mm or between 2 and 3 mm.

The terminal filter element (at the downstream end of the article/consumable) may be joined to the upstream elements forming the article/consumable by a circumscribing tipping layer e.g. a tipping paper layer. The tipping paper may have an axial length longer than the axial length of the terminal filter element such that the tipping paper completely circumscribes the terminal filter element plus the wrapping layer surrounding any adjacent upstream element.

The or at least one of the filter elements e.g. the terminal filter element may include a capsule e.g. a crushable capsule (crush-ball) containing a liquid flavourant e.g. any of the flavourants listed above. The capsule can be crushed by the user during smoking of the article/consumable to release the flavourant. The capsule may be located at the axial centre of the terminal filter element.

In some embodiments, the second section of the article/consumable may comprise an aerosol-cooling element which is adapted to cool the aerosol generated from the aerosol-forming substrate (by heat exchange) before being inhaled by the user.

The aerosol-cooling element will be downstream from the aerosol-forming substrate. For example, it may be between the aerosol-forming substrate and a/the filter element and/or between two filter elements. The aerosol cooling element may be at least partly (e.g. completely) circumscribed by the (paper) wrapping layer.

The aerosol-cooling element may be formed of a plastics material selected from the group consisting of polylactic acid (PLA), polyvinyl chloride (PVC), polyethylene (PE) and polyethylene terephthalate (PET). The aerosol-cooling element may be formed of a crimped/gathered sheet of material to form a structure having a high surface area with a plurality of longitudinal channels to maximise heat exchange and cooling of the aerosol.

The second section of the article/consumable may comprise a spacer element that defines a space or cavity or chamber between the aerosol-forming substrate and the downstream end of the article/consumable. The spacer acts to allow both cooling and mixing of the aerosol. It may be provided between the aerosol-forming substrate and a/the filter element and/or between two filter elements. The spacer element may comprise a cardboard tube. The spacer element may be at least partly (e.g. entirely) circumscribed by the (paper) wrapping layer.

In some embodiments the second section comprises one or more filter elements and a spacer element.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In some embodiments, the article/consumable is frangible to separate the first and second sections. This provides an article which is much easier for the user to separate into discrete first and second sections after use. In some embodiments, the consumable is frangible by breaking along a region of weakness between the first section and the second section. In some embodiments, the region of weakness comprises a plurality of perforations in the outer wrapping layer of the consumable. In some embodiments, the region of weakness includes a line of weakness in the outer wrapping layer of the consumable. In some embodiments, this line of weakness is provided by an annular array of perforations in the outer wrapping layer. The line of weakness/array of perforations will be aligned with the junction between the first and second sections i.e. at the axially downstream end of the aerosol-forming substrate.

In a second aspect of the fifth mode, there is provided a kit comprising an aerosol-forming article according to the first aspect of the fifth mode, and a package comprising a first housing and a second housing, the package being adapted to house one or more aerosol-forming articles according to the first aspect of the fifth mode in the first housing, and adapted to house one or more of the second sections of an aerosol-forming article according to the first aspect of the fifth mode in the second housing after separation of the second section from the first section.

The kit comprises a package comprising a first housing and a second housing. The first housing is adapted to house one or more aerosol-forming articles according to the first aspect of the fifth mode, i.e. unused articles which may be removed from the housing and inserted into a smoking substitute system for use by the user. The first housing may define a recess or cavity of sufficient dimensions to house one or more articles, for example at least 5 or at least 10 articles. The first housing may be adapted to completely contain/cover any articles in a first configuration and be movable into a second configuration in which one or more articles are revealed and removable from the housing by the user. For example the package may comprise a removable flap or lid to cover any articles within the first housing.

The second housing is adapted to house one or more of the second sections of an aerosol-forming article according to the first aspect of the fifth mode, after the user has separated the article into first and second sections and disposed of the first section (for example, in an ashtray). The second housing may define a recess or cavity of sufficient dimensions to house one or more second sections of articles, for example at least 5 or at least 10 articles. In some embodiments the number of articles which can be housed by the first housing is equal to the number of second sections of articles which can be housed by the second housing. The second housing may be adapted to completely contain/cover any sections in a first configuration and be movable into a second configuration in which one or more sections are revealed and removable from the housing by the user. For example, the package may comprise a removable flap or lid to cover any sections within the first housing.

The presence of the second housing provides a convenient means for the user to transport the used second sections of articles after use, for example for later disposal at home, for example disposal by recycling. After an article has been used, the user can separate the article into first and second sections and dispose of the first section immediately, for example in an ashtray or by composting. The first section of the article can then be returned to the package from which the article was taken, but placed into a distinct second housing adapted to house the used second sections of articles. In this way the unused, whole articles are kept separate from the used second sections of articles within the package. At a later time the user can retrieve the second sections from the second housing of the package for disposal, for example by recycling. Alternatively the user can leave the second sections inside the second housing until the first housing is empty and the second housing is full, before disposing of the entire package with the second sections of articles inside. A simple, clean means for disposal is therefore provided.

In a third aspect of the fifth mode, there is provided a smoking substitute system comprising an aerosol-forming article according to the first aspect of the fifth mode and a device comprising a heating element.

The device may be a HNB device i.e. a device adapted to heat but not combust the aerosol-forming substrate.

The device may comprise a main body for housing the heating element. The heating element may comprise an elongated e.g. rod, tube-shaped or blade heating element. The heating element may project into or surround a cavity within the main body for receiving the article/consumable described above.

The device (e.g. the main body) may further comprise an electrical power supply e.g. a (rechargeable) battery for powering the heating element. It may further comprise a control unit to control the supply of power to the heating element.

In a fourth aspect of the fifth mode, there is provided a method of using a smoking substitute system according to the second aspect, the method comprising: inserting the article/consumable into the device; and heating the article/consumable using the heating element.

In some embodiments, the method comprises inserting the article/consumable into a cavity within the main body and penetrating the article/consumable with the heating element upon insertion of the article/consumable. For example, the heating element may penetrate the aerosol-forming substrate in the article/consumable.

The method may further comprise separating the first and second sections of the article after heating.

The skilled person will appreciate that except where mutually exclusive, a feature or parameter described in relation to any one of the above aspects of the fifth mode may be applied to any other aspect. Furthermore, except where mutually exclusive, any feature or parameter of the fifth mode described herein may be applied to any aspect of the fifth mode and/or combined with any other feature or parameter of the fifth mode described herein.

Sixth Mode of the Disclosure: Biodegradable Materials

At its most general, the sixth mode of the present disclosure relates to an aerosol-forming article e.g. a smoking substitute article such as an HNB consumable which is disposable in a more environmentally-friendly manner.

According to a first aspect of the sixth mode, there is provided an aerosol-forming article (e.g. a smoking substitute article such as an HNB consumable) which comprises solely biodegradable materials.

The inventors have found that existing aerosol-forming articles, such as existing HNB consumables, contain at least one non-biodegradable or non-readily-biodegradable element and are therefore difficult to dispose of in an environmentally-friendly manner. For example, aerosol-forming articles may comprise non-biodegradable plastics materials, non-biodegradable inks, non-biodegradable paper (due to the presence of non-biodegradable constituents within the paper such as inorganic fillers), non-biodegradable adhesives (due to the presence of non-biodegradable polymers within the adhesives) and other non-biodegradable components which necessitate disposal by traditional, non-environmentally friendly means such as landfill.

In providing an aerosol-forming article which is entirely biodegradable, the disposal of the aerosol-forming article after use by alternative, more environmentally-friendly means, is facilitated, for example disposal by composting.

Herein, the term “biodegradable” takes its usual meaning, indicating a material which is capable of being degraded or decomposed by bacteria, fungi or other living organisms. Standard cellulose acetate tow filters used in aerosol-forming articles such as cigarettes and HNB consumables have a degree of acetyl substitution (DS) of 2.5 which provides good solubility in common solvents, good molecular weight and good melt properties. However, such cellulose acetate filter tow is not readily biodegradable, requiring lengthy time periods to achieve even partial disintegration under natural composting conditions. Thus cellulose acetate is not considered to be “readily biodegradable” in the context of the present invention. In some embodiments, the term “readily biodegradable” indicates a material which is at least 30 wt % degraded after 46 days as determined according to the ISO 11734 standard of 1995 (anaerobic biodegradability in municipal wastewater treatment plant sludge at 35° C.), for example at least 35 wt % degraded, at least 40 wt % degraded, at least 45 wt % degraded or at least 50 wt % degraded. Cellulose acetate fibers of the type used in cellulose acetate tow filters (DS=2.45) are 27 wt % degraded after 46 days as determined according to the ISO 11734 standard (Gartiser et al., “Assessment of Several Test Methods for the Determination of the Anaerobic Biodegradability of Polymers”, J. Polymer Environ., Vol. 6, No. 3, 1998, pp. 159-173).

Optional features will now be set out. These are applicable singly or in any combination with any aspect of the sixth mode.

The aerosol-forming article is preferably a heat-not-burn (HNB) consumable.

The aerosol-forming article preferably comprises an aerosol-forming substrate

The aerosol-forming substrate is capable of being heated to release at least one volatile compound that can form an aerosol. The aerosol-forming substrate may be located at the upstream end of the article/consumable.

As used herein, the terms “upstream” and “downstream” are intended to refer to the flow direction of the vapour/aerosol i.e. with the downstream end of the article/consumable being the mouth end or outlet where the aerosol exits the article/consumable for inhalation by the user. The upstream end of the article/consumable is the opposing end to the downstream end.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

Preferably, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above-mentioned tobaccos.

Any suitable parts of the tobacco plant may be used. This includes leaves, stems, roots, bark, seeds and flowers.

The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon).

The aerosol-forming substrate may comprise a gathered sheet of homogenised (e.g. paper/slurry recon) tobacco or gathered shreds/strips formed from such a sheet.

In some embodiments, the sheet used to form the aerosol-forming substrate has a grammage greater than or equal to 100 g/m², e.g. greater than or equal to 110 g/m² such as greater than or equal to 120 g/m².

The sheet may have a grammage of less than or equal to 300 g/m² e.g. less than or equal to 250 g/m² or less than or equal to 200 g/m².

The sheet may have a grammage of between 120 and 190 g/m².

The aerosol-forming substrate may comprise at least 50 wt % plant material, e.g. at least 60 wt % plant material e.g. around 65 wt % plant material. The aerosol-forming substrate may comprise 80 wt % or less plant material e.g. 75 or 70 wt % or less plant material. The aerosol-forming substrate may comprise from 50 to 80 wt % plant material, for example from 50 to 75 wt %, from 55 to 80 wt %, from 55 to 75 wt %, from 50 to 70 wt %, from 55 to 70 wt %, from 60 to 75 wt % or from 60 to 70 wt %.

The aerosol-forming substrate may comprise one or more additives selected from humectants, flavourants, biodegradable fillers, aqueous/non-aqueous solvents and biodegradable binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %.

Suitable binders are known in the art and may act to bind together the components forming the aerosol-forming substrate. Binders may comprise starches and/or cellulosic binders such as methyl cellulose, ethyl cellulose, hydroxypropyl cellulose, hydroxyethyl cellulose and methyl cellulose, gums such as xanthan, guar, arabic and/or locust bean gum, organic acids and their salts such as alginic acid/sodium alginate, agar and pectins.

Preferably the binder content is 5 to 10 wt % of the aerosol-forming substrate e.g. around 6 to 8 wt %.

Suitable fillers are known in the art and may act to strengthen the aerosol-forming substrate. Fillers may comprise fibrous (non-tobacco) fillers such as cellulose fibres, lignocellulose fibres (e.g. wood fibres), jute fibres and combinations thereof.

Preferably, the filler content is 5 to 10 wt % of the aerosol-forming substrate e.g. around 6 to 9 wt %.

The aerosol-forming substrate may comprise an aqueous and/or non-aqueous solvent. In some embodiments, the aerosol forming substrate has a water content of between 5 and 10 wt % e.g. between 6-9 wt % such as between 7-9 wt %.

The flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g. between 6 and 9 mm or 6 and 8 mm e.g. around 7 mm. It may have an axial length of between 10 and 15 mm e.g. between 11 and 14 mm such as around 12 or 13 mm.

The aerosol-forming substrate may be circumscribed by a biodegradable (e.g. readily biodegradable) wrapping layer.

In some embodiments, the wrapping layer comprises one or more materials selected from biodegradable paper/cardboard, plant material (e.g. tobacco), biodegradable polymer/plastics material (e.g. polylactic acid), cellulosic/lignocellulosic material (e.g. wood, bamboo, jute). Any inks or adhesives used with the wrapping layer will be biodegradable.

The wrapping layer does not comprise non-biodegradable inks, fillers or adhesives.

The article/consumable may comprise at least one filter element. There may be a terminal filter element at the downstream/mouth end of the article/consumable. There may be an upstream filter element upstream from the terminal filter element. The terminal filter element and upstream filter element may be axially adjacent or may be axially spaced.

The filter element(s) is/are biodegradable, preferably readily biodegradable.

In some embodiments, the filter element(s) comprise(s) one or more materials selected from biodegradable paper/cardboard, plant material (e.g. tobacco such as extruded tobacco), biodegradable polymer/plastics material (e.g. polylactic acid) and cellulosic/lignocellulosic material (e.g. wood, bamboo, jute). The filter element(s) do not comprise cellulose acetate.

The or each filter element may be circumscribed with a plug wrap e.g. a biodegradable paper plug wrap.

The or each filter element may have a substantially cylindrical shape with a diameter substantially matching the diameter of the aerosol-forming substrate (with or without its associated wrapping layer). The axial length of the or each filter element may be less than 20 mm, e.g. between 8 and 15 mm, for example between 9 and 13 mm e.g. between 10 and 12 mm.

The or at least one of the filter element(s) may be a solid filter element. The or at least one of the filter element(s) may be a hollow bore filter element. The or each hollow bore filter element may have a bore diameter of between 1 and 5 mm, e.g. between 2 and 4 mm or between 2 and 3 mm.

The upstream filter element may be at least partly (e.g. entirely) circumscribed by the (biodegradable) wrapping layer.

The terminal filter element (at the downstream end of the article/consumable) may be joined to the upstream elements forming the article/consumable by a circumscribing biodegradable tipping layer e.g. a tipping paper layer. The tipping paper may have an axial length longer than the axial length of the terminal filter element such that the tipping paper completely circumscribes the terminal filter element plus the wrapping layer surrounding any adjacent upstream element.

The or at least one of the filter elements e.g. the terminal filter element may include a biodegradable capsule e.g. a crushable capsule (crush-ball) containing a liquid flavourant e.g. any of the flavourants listed above.

The capsule can be crushed by the user during smoking of the article/consumable to release the flavourant. The capsule may be located at the axial centre of the terminal filter element.

In some embodiments, the article/consumable may comprise a biodegradable (e.g. readily biodegradable) aerosol-cooling element. The aerosol-cooling element is adapted to cool the aerosol generated from the aerosol-forming substrate (by heat exchange) before being inhaled by the user.

The aerosol-cooling element will be downstream from the aerosol-forming substrate. For example, it may be between the aerosol-forming substrate and the upstream filter element and/or between two filter elements. The aerosol cooling element may be at least partly (e.g. completely) circumscribed by the (paper) wrapping layer.

In some embodiments, the biodegradable aerosol-cooling element comprises one or more materials selected from biodegradable paper/cardboard, plant material (e.g. tobacco, such as extruded tobacco), biodegradable polymer/plastics material (e.g. polylactic acid) and cellulosic/lignocellulosic material (e.g. wood, bamboo, jute). The cooling element does not comprise cellulose acetate.

The aerosol-cooling element may be formed of a biodegradable plastics material such as polylactic acid (PLA). The aerosol-cooling element may be formed of a crimped/gathered sheet of biodegradable material to form a structure having a high surface area with a plurality of longitudinal channels to maximise heat exchange and cooling of the aerosol.

The article/consumable may comprise a biodegradable (e.g. readily biodegradable) spacer element that defines a space or cavity or chamber between the aerosol-forming substrate and the downstream end of the article/consumable. For example, it may be between the aerosol-forming substrate and the upstream filter element and/or between the two filter elements. The spacer acts to allow both cooling and mixing of the aerosol.

In some embodiments the biodegradable spacer element comprises one or more materials selected from biodegradable paper/cardboard, plant material (e.g. tobacco, such as extruded tobacco), biodegradable polymer/plastics material (e.g. polylactic acid) and cellulosic/lignocellulosic material (e.g. wood, bamboo, jute). The spacer element does not comprise cellulose acetate.

The spacer element may comprise a tubular element e.g. a biodegradable cardboard tube or an extruded tube of plant material e.g. tobacco. The spacer element may be at least partly (e.g. entirely) circumscribed by the (paper) wrapping layer.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

The article/consumable does not include any components which comprise polymer materials selected from polyethylene, polypropylene, polyvinylchloride, polystyrene, polyether ether ketone (PEEK) or polytetrafluoroethylene. Such polymer materials are non-biodegradable, so an article/consumable which does not contain any of them is easier to dispose of in an environmentally-friendly manner.

In some embodiments, the article/consumable comprises one or more filter elements (e.g. two filter elements), a spacer element, an aerosol-forming substrate, a wrapping layer and a tipping layer, each of which comprises one or more materials selected from biodegradable cardboard/paper, plant material (e.g. tobacco), biodegradable polymer/plastics material (e.g. polylactic acid), cellulosic/lignocellulosic material (e.g. wood, bamboo, jute). Any inks or adhesives used in the article/consumable are biodegradable.

In some embodiments, each component of the article/consumable (including the aerosol-forming substrate, filter element(s), wrapping layer, tipping paper layer, aerosol cooling element and spacer element) is composed of materials which are at least 30 wt % degraded after 46 days as determined according to the ISO 11734 standard of 1995 (anaerobic biodegradability in municipal wastewater treatment plant sludge at 35° C.), for example at least 35 wt % degraded, at least 40 wt % degraded, at least 45 wt % degraded or at least 50 wt % degraded. Cellulose acetate fibers of the type used in cellulose acetate tow filters (DS=2.45) are 27 wt % degraded after 46 days as determined according to the ISO 11734 standard.

In some embodiments, the entire article/consumable is completely disintegrated after a time period of 14 days or less under natural composting conditions, for example 13 days or less, 12 days or less, 11 days or less or 10 days or less.

The article/consumable does not contain any components which comprise cellulose acetate.

In some embodiments, the article/consumable comprises a region of weakness between the aerosol-forming substrate and the downstream components i.e. there may be a region of weakness at the downstream axial end of the aerosol-forming substrate e.g. between the aerosol-forming substrate and the upstream filter element. In this way, different sections of the article/consumable may be disposed of in different ways. For example, the user may wish to dispose of the aerosol-forming substrate into an ashtray while disposing of the downstream elements in an alternative manner, for example by composting. Separation of the consumable into multiple separate sections after use is much easier due to the provision of the region of weakness in a specific location.

In some embodiments, the region of weakness comprises a discrete region of the wrapping layer which is of lower grammage than the remaining wrapping layer. In some embodiments, the region of weakness comprises an array of perforations in the wrapping layer. The array of perforations may extend around the entire circumference of the consumable. In some embodiments, the region of weakness comprises a linear circular array of perforations extending around the circumference of the consumable.

In a second aspect of the sixth mode, there is provided a smoking substitute system comprising an aerosol-forming article according to the first aspect and a device comprising a heating element.

The device may be a HNB device i.e. a device adapted to heat but not combust the aerosol-forming substrate.

The device may comprise a main body for housing the heating element. The heating element may comprise an elongated e.g. rod, tube-shaped or blade heating element. The heating element may project into or surround a cavity within the main body for receiving the article/consumable described above.

The device (e.g. the main body) may further comprise an electrical power supply e.g. a (rechargeable) battery for powering the heating element. It may further comprise a control unit to control the supply of power to the heating element.

In a third aspect of the sixth mode, there is provided a method of using a smoking substitute system according to the second aspect, the method comprising: inserting the article/consumable into the device; and heating the article/consumable using the heating element.

In some embodiments, the method comprises inserting the article/consumable into a cavity within the main body and penetrating the article/consumable with the heating element upon insertion of the article/consumable. For example, the heating element may penetrate the aerosol-forming substrate in the article/consumable.

The skilled person will appreciate that except where mutually exclusive, a feature or parameter of the sixth mode described in relation to any one of the above aspects of the sixth mode may be applied to any other aspect of the sixth mode. Furthermore, except where mutually exclusive, any feature or parameter described herein may be applied to any aspect of the sixth mode and/or combined with any other feature or parameter of the sixth mode described herein.

Seventh Mode of the Disclosure: Aerosol-Forming Article Having an Improved Cooling Element

At its most general, the seventh mode of the present disclosure relates to i) an aerosol-forming article e.g. an HNB consumable having an increased concentration of nicotine, and ii) an aerosol-forming article e.g. a smoking substitute article such as an HNB consumable having a cooling element that acts to increase the volatile compound concentration of the vapour.

According to a first aspect of the seventh mode, the present disclosure provides an aerosol-forming article (e.g. an HNB consumable) comprising an aerosol-forming substrate dosed with nicotine and/or a nicotine salt, wherein the aerosol-forming substrate is a slurry-type reconstituted plant material.

Reference to “slurry-type” corresponds to a reconstituted plant material made by slurry-casting as opposed to a paper-making process.

By using an aerosol-forming substrate (which will itself include a volatile compound) dosed with nicotine and/or a nicotine salt, the number/concentration of volatile compound(s) can be increased to provide the user with a vapour/aerosol containing a higher concentration of the volatile compound(s). This may provide the user with an enhanced recreational and/or medicinal effect when inhaled. In particular, the amount of nicotine in the consumable is increased. This improves the “nicotine” hit experienced by the user.

Preferably, the aerosol-forming substrate is dosed with a nicotine salt.

Advantageously, nicotine salts exhibit lower volatility and higher stability than nicotine, therefore provide a safer and more convenient means of handling during processing.

According to a second aspect of the seventh mode, there is provided an aerosol-forming article (e.g. a smoking substitute article such as an HNB consumable) comprising an aerosol-forming substrate and a cooling element wherein the cooing element is adapted to increase the concentration of a volatile compound within a vapour generated by heating of the aerosol-forming substrate.

By providing a cooling element that not only cools a vapour generated by heating of the aerosol-forming substrate, also increases the concentration of a volatile compound within the vapour, the recreational and/or medicinal effect achieved by the user upon inhalation of the vapour is enhanced.

The term “nicotine salt” is used to define an ionic compound formed by the reaction between nicotine and a proton donor e.g. by the reaction between nicotine and an acid. Accordingly, the nicotine salt may be an acid salt.

Optional features will now be set out. These are applicable singly or in any combination with any aspect of the seventh mode.

The aerosol-forming article is preferably a heat-not-burn (HNB) consumable.

The aerosol-forming substrate is capable of being heated to release at least one volatile compound that can form an aerosol. The aerosol-forming substrate may be located at the upstream end of the article/consumable.

In some embodiments, in order for the aerosol-cooling element to increase the concentration of the volatile compound in the vapour, the aerosol-cooling element comprises at least one volatile compound additive. The at least one volatile compound additive is intended to be vaporised/aerosolised as vapour from the aerosol-forming substrate passes through the cooling element. Suitable chemical and/or physiologically active volatile compound additives include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing.

The at least one volatile compound additive may comprise nicotine and/or nicotine salts.

The aerosol-cooling element is downstream from the aerosol-forming substrate. For example, it may be between the aerosol-forming substrate and a/the filter element and/or between two filter elements. The aerosol cooling element may be at least partly (e.g. completely) circumscribed by the (paper) wrapping layer.

As used herein, the terms “upstream” and “downstream” are intended to refer to the flow direction of the vapour/aerosol i.e. with the downstream end of the article/consumable being the mouth end or outlet where the aerosol exits the article/consumable for inhalation by the user. The upstream end of the article/consumable is the opposing end to the downstream end.

The aerosol-cooling element may be formed of a plastics material selected from the group consisting of polylactic acid (PLA), polyvinyl chloride (PVC), polyethylene (PE) and polyethylene terephthalate (PET). The aerosol-cooling element may be formed of a crimped/gathered sheet of material (e.g. plastic material) to form a structure having a high surface area with a plurality of longitudinal channels to maximise heat exchange and cooling of the aerosol.

The sheet of material may be sprayed with a solution of the volatile compound additive prior to crimping/gathering. Alternatively, the cooling element may be soaked in or impregnated with a solution of the volatile compound additive.

The aerosol-forming substrate may be located at the upstream end of the article/consumable.

The aerosol-forming substrate is capable of being heated to release at least one volatile compound that can form an aerosol. The volatile compound may be the same as or different to the volatile compound additive in the cooling element.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A, together with any combinations, functional equivalents to, and/or synthetic alternatives of the foregoing.

Preferably, the nicotine salt is selected from nicotine hydrochloride, nicotine dihydrochloride, nicotine monotartrate, nicotine bitartrate, nicotine bitartrate dihydrate, nicotine benzoate, nicotine fumarate, nicotine orotate, nicotine sulphate, nicotine zinc chloride monohydrate, nicotine lactate and nicotine salicylate, and combinations thereof.

The aerosol-forming substrate comprises plant material. The plant material may comprise at least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

Preferably, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon).

The aerosol-forming substrate may comprise a gathered sheet of homogenised slurry recon tobacco or gathered shreds/strips formed from such a sheet.

In some embodiments, the sheet used to form the aerosol-forming substrate has a grammage greater than or equal to 100 g/m², e.g. greater than or equal to 110 g/m² such as greater than or equal to 120 g/m².

The sheet may have a grammage of less than or equal to 300 g/m² e.g. less than or equal to 250 g/m² or less than or equal to 200 g/m².

The sheet may have a grammage of between 120 and 190 g/m².

In some embodiments, the sheet used to form the aerosol-forming substrate has a density of less than 1100 mg/cm³, preferably less than 1000 mg/cm³, and more preferably less than 900 mg/cm³.

In some embodiments, the aerosol-forming substrate has a mass/surface area of less than 0.09 mg/mm², preferably less than 0.085 mg/mm², preferably less than 0.08 mg/mm² and more preferably less than 0.075 mg/mm².

In some embodiments the aerosol-forming substrate may have a thickness less than about 1 mm, preferably less than about 0.5 mm, preferably between about 0.1 and 0.3 mm, such as about 0.2 mm.

The aerosol-forming substrate may comprise at least 50 wt % plant material, e.g. at least 60 wt % plant material e.g. around 65 wt % plant material. The aerosol-forming substrate may comprise 80 wt % or less plant material e.g. 75 or 70 wt % or less plant material. The aerosol-forming substrate may comprise one or more additives selected from humectants, flavourants, fillers, aqueous/non-aqueous solvents and binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %.

Suitable binders are known in the art and may act to bind together the components forming the aerosol-forming substrate. Binders may comprise starches and/or cellulosic binders such as methyl cellulose, ethyl cellulose, hydroxypropyl cellulose, hydroxyethyl cellulose and carboxymethyl cellulose, gums such as xanthan, guar, arabic and/or locust bean gum, organic acids and their salts such as alginic acid/sodium alginate, agar and pectins.

Preferably the binder content is 5 to 20 wt % of the aerosol-forming substrate e.g. around 5 to 10 wt % or around 6 to 8 wt %. The upper limit of binder is preferably up to 15 wt. %, such as up to 10 wt. %. Preferably the binder content is above 5 wt %.

Suitable fillers are known in the art and may act to strengthen the aerosol-forming substrate. Fillers may comprise fibrous (non-tobacco) fillers such as cellulose fibres, lignocellulose fibres (e.g. wood fibres), jute fibres and combinations thereof.

Preferably, the filler content is 5 to 10 wt % of the aerosol-forming substrate e.g. around 6 to 9 wt %.

The aerosol-forming substrate may comprise an aqueous and/or non-aqueous solvent. In some embodiments, the aerosol forming substrate has a water content of between 5 and 10 wt % e.g. between 6-9 wt % such as between 7-9 wt %.

Suitable flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g. between 6 and 9 mm or 6 and 8 mm e.g. around 7 mm. It may have an axial length of between 10 and 15 mm e.g. between 11 and 14 mm such as around 12 or 13 mm.

The aerosol-forming substrate may be circumscribed by a wrapping layer e.g. a paper wrapping layer. The wrapping layer may overlie an inner foil layer or may comprise a paper/foil laminate (with the foil innermost).

The article/consumable may comprise at least one filter element. There may be a terminal filter element at the downstream/mouth end of the article/consumable. There may be a plurality of filter elements (e.g. the terminal filter element and an upstream filter element) which may be adjacent one another or which may be spaced apart. Any upstream filter element may be at least partly (e.g. entirely) circumscribed by the (paper) wrapping layer.

The or at least one of the filter element(s) (e.g. the terminal filter element and/or upstream filter element) may be comprised of cellulose acetate or polypropylene tow. The or at least one of the filter element(s) (e.g. the terminal filter element and/or upstream filter element may be comprised of activated charcoal. The or at least one of the filter element(s) (e.g. the terminal filter element and/or upstream filter element may be comprised of paper. The or at least one of the filter element(s) (e.g. the terminal filter element and/or upstream filter element may be comprised of plant material e.g. extruded plant material. The or each filter element may be circumscribed with a plug wrap e.g. a paper plug wrap.

The or each filter element may have a substantially cylindrical shape with a diameter substantially matching the diameter of the aerosol-forming substrate (with or without its associated wrapping layer). The axial length of the or each filter element may be less than 20 mm, e.g. between 8 and 15 mm, for example between 9 and 13 mm e.g. between 10 and 12 mm.

The or at least one of the filter element(s) may be a solid filter element. The or at least one of the filter element(s) may be a hollow bore filter element. The or each hollow bore filter may have a bore diameter of between 1 and 5 mm, e.g. between 2 and 4 mm or between 2 and 3 mm.

There may be a plurality of e.g. two filter elements which may be adjacent one another or which may be spaced apart. Any filter element(s) upstream of the terminal filter element may be at least partly (e.g. entirely) circumscribed by the (paper) wrapping layer.

The terminal filter element (at the downstream end of the article/consumable) may be joined to the upstream elements forming the article/consumable by a circumscribing tipping layer e.g. a tipping paper layer. The tipping paper may have an axial length longer than the axial length of the terminal filter element such that the tipping paper completely circumscribes the terminal filter element plus the wrapping layer surrounding any adjacent upstream element.

The or at least one of the filter elements e.g. the terminal filter element may include a capsule e.g. a crushable capsule (crush-ball) containing a liquid flavourant e.g. any of the flavourants listed above. The capsule can be crushed by the user during smoking of the article/consumable to release the flavourant. The capsule may be located at the axial centre of the terminal filter element.

In some embodiments, the article/consumable may comprise an aerosol-cooling element which is adapted to cool the aerosol generated from the aerosol-forming substrate (by heat exchange) before being inhaled by the user.

The aerosol-cooling element will be downstream from the aerosol-forming substrate. For example, it may be between the aerosol-forming substrate and the upstream filter element and/or between the two filter elements. The aerosol cooling element may be at least partly (e.g. completely) circumscribed by the (paper) wrapping layer.

The aerosol-cooling element may be formed of a plastics material selected from the group consisting of polylactic acid (PLA), polyvinyl chloride (PVC), polyethylene (PE) and polyethylene terephthalate (PET). The aerosol-cooling element may be formed of a crimped/gathered sheet of material to form a structure having a high surface area with a plurality of longitudinal channels to maximise heat exchange and cooling of the aerosol.

The article/consumable may comprise a spacer element that defines a space or cavity between the aerosol-forming substrate and the downstream end of the article/consumable. It may be provided between the aerosol-forming substrate and the upstream filter element and/or between the two filter elements. The spacer element may comprise a tubular element e.g. a cardboard tube. The spacer element may be at least partly (e.g. entirely) circumscribed by the (paper) wrapping layer. The spacer acts to allow both cooling and mixing of the aerosol.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In a third aspect of the seventh mode, there is provided a smoking substitute system comprising an aerosol-forming article according to the first aspect or the second aspect, and a device comprising a heating element.

The device may be a HNB device i.e. a device adapted to heat but not combust the aerosol-forming substrate.

The device may comprise a main body for housing the heating element. The heating element may comprise an elongated e.g. rod, tube-shaped or blade heating element. The heating element may project into or surround a cavity within the main body for receiving the article/consumable described above.

The device (e.g. the main body) may further comprise an electrical power supply e.g. a (rechargeable) battery for powering the heating element. It may further comprise a control unit to control the supply of power to the heating element.

In a fourth aspect of the seventh mode, there comprises a method of using a smoking substitute system according to the third aspect, the method comprising: inserting the article/consumable into the device; and heating the article/consumable using the heating element.

In some embodiments, the method comprises inserting the article/consumable into a cavity within the main body and penetrating the article/consumable with the heating element upon insertion of the article/consumable. For example, the heating element may penetrate the aerosol-forming substrate in the article/consumable.

In a fifth aspect of the seventh mode, there is provided a method for manufacturing an aerosol-forming substrate, wherein the aerosol forming substrate is a slurry-type reconstituted plant material comprising: combining a ground plant material with an aqueous medium to form a slurry; and combining nicotine and/or a nicotine salt with the slurry. Preferably, a nicotine salt is combined with the slurry.

Advantageously, by combining nicotine and/or a nicotine salt with the slurry a greater degree of homogeneity of the nicotine and the plant material is achieved. This provides the user with an enhanced and more consistent user experience. Also, the amount of nicotine can be more accurately controlled and tailored to the user's requirements, thus more easily enabling the production of bespoke blends.

The nicotine and/or nicotine salt can be added to the slurry in combination with other additives or alone. In particular, the nicotine and/or nicotine salt may be combined with the tobacco prior to or following grinding. Alternatively, the nicotine and/or nicotine salt may be combined with the pulp mix.

There is no particular limitation as to when the nicotine and/or nicotine salt is added to the slurry (i.e. it can be added at any stage of slurry processing) as long as it is present in the slurry rather than, for example, being sprayed onto a cast sheet of slurry mixture.

It is preferable that the plant material is ground to a particle size of less than 120 microns. It is undesirable to have a particle size greater than 150 microns as this is associated with the formation of defects and inhomogeneous areas in the sheet formed by casting the slurry.

Particularly, preferred is a particle size of 30 to 120 microns.

A binder may also be included within the slurry. The amount of binder used is chosen to provide an amount of 5 to 20 weight % of binder in the sheet ultimately produced from the slurry, such as 5 to 15 weight %, such as 5 to 10 weight %. The ground tobacco may be combined with the binder before combination with the aqueous medium. Or alternatively, the binder may be combined with the aqueous medium prior to combining the aqueous medium with the ground tobacco.

The slurry may also contain fillers, humectants, preservatives, and/or flavours. These may be combined with the ground plant material before combination with the aqueous medium, or alternatively can be added to the slurry separately

It is preferred that the total solids content of the slurry is between 15% and 30% of the total slurry weight. Of this preferred range about 50 to 90% of the total solids content of the slurry should be the plant material. The plant material content of the slurry is not critical, but it is preferred that the minimum amount of aqueous medium is used to avoid leaching of plant material solubles into the aqueous medium.

The slurry is formed by mixing the various components of the slurry. This may be achieved using a high energy mixer or a high shear mixer to homogenize the slurry. During mixing the slurry may optionally be heated.

Additionally, the slurry may be vibrated to aid homogenization of the slurry. This may be achieved by vibrating a tank or silo in which the slurry is contained.

After mixing the slurry is cast onto a moving support surface, such as a dryer belt to form a sheet. Casting of the sheet may be achieved by a roll coater system or a laminator, or any other method suitable in the art. It is preferred that the moisture content of the sheet at casting is between 60 and 80% of the total weight of the cast slurry. The cast sheet is then dried by exposure to elevated temperatures and/or reduced pressures to remove excess moisture content from the sheet. Suitable drying methods include a steam dryer and/or heated air dryers. Alternatively, the sheet may be dried under ambient conditions,

Preferably, the nicotine salt is selected from nicotine hydrochloride, nicotine dihydrochloride, nicotine monotartrate, nicotine bitartrate, nicotine bitartrate dihydrate, nicotine benzoate, nicotine fumarate, nicotine orotate, nicotine sulphate, nicotine zinc chloride monohydrate, nicotine lactate and nicotine salicylate, and combinations thereof.

Advantageously, these salts provide a satisfactory “nicotine hit”. Also, by having lower volatility and higher stability in comparison to nicotine these salts are easier to handle during processing of the slurry.

Preferably, the nicotine and/or a nicotine salt is provided as an aqueous solution.

Advantageously, the non-toxicity and non-flammability of water minimises handling risks during processing. Furthermore, the “premixing” of the nicotine salt with a solvent prior to combination with the slurry, improves the homogeneous nature of the nicotine salt and the slurry mixture.

Preferably, the plant material is tobacco.

As the disclosure is primarily intended to provide a smoking consumable, the use of tobacco provides a slurry-type reconstituted plant material having the inherent properties of tobacco, for examples, the texture, aroma and presence of nicotine.

The method according the fifth aspect of the seventh mode may comprise a step of winding the formed sheet onto a bobbin and/or a step of making an HNB consumable.

According to a sixth aspect of the seventh mode there is provided an aerosol-forming substrate for an HNB device produced by the process according to the fifth aspect of the seventh mode.

Preferably, the aerosol-forming substrate according to the sixth aspect of the seventh mode comprises a nicotine salt selected from nicotine hydrochloride, nicotine dihydrochloride, nicotine monotartrate, nicotine bitartrate, nicotine bitartrate dihydrate, nicotine benzoate, nicotine fumarate, nicotine orotate, nicotine sulphate, nicotine zinc chloride monohydrate nicotine lactate and nicotine salicylate, and combinations thereof, wherein the nicotine salt content is 2 to 15% by weight of the reconstituted tobacco product.

The skilled person will appreciate that except where mutually exclusive, a feature or parameter of the seventh mode described in relation to any one of the above aspects of the seventh mode may be applied to any other aspect of the seventh mode. Furthermore, except where mutually exclusive, any feature or parameter of the seventh mode described herein may be applied to any aspect and/or combined with any other feature or parameter of the seventh mode described herein.

Eighth Mode of the Disclosure: Adaption Providing Greater Ease of Use.

At its most general, the eighth mode of the present disclosure relates to an aerosol-forming article e.g. a smoking substitute article such as an HNB consumable which includes an adaptation providing greater ease of use, in particular greater ease of controlling the movement, position and/or location of the consumable within an external body or housing (for example, a housing associated with a heating device). A smoking substitute consumable, such as a HNB consumable, is provided which includes means to limit movement of the consumable relative to an external body, such as a heating device.

According to a first aspect of the eighth mode of the disclosure, there is provided an aerosol-forming article (e.g. heat not burn (HNB) consumable) having an outer surface comprising at least one laterally- or radially-extending projection adapted to engage with a heating device.

The laterally-/radially-extending projection serves to engage with a heating device in order to allow accurate control of the positioning of the article within the device. This ensures that the position of the article within the device is correct to facilitate optimum heating of the article and optimum mouthpiece position for the user. Furthermore, the projection can serve to seal the article within a chamber of a device to prevent undesirable air flow around the outer surface of the article.

Optional features will now be set out. These are applicable singly or in any combination with any aspect of the eighth mode. The aerosol-forming article is preferably a heat-not-burn (HNB) consumable.

The article comprises an aerosol-forming substrate that is capable of being heated to release at least one volatile compound that can form an aerosol. The aerosol-forming substrate may be located at the upstream end of the article/consumable.

As used herein, the terms “upstream” and “downstream” are intended to refer to the flow direction of the vapour/aerosol i.e. with the downstream end of the article/consumable being the mouth end or outlet where the aerosol exits the article/consumable for inhalation by the user. The upstream end of the article/consumable is the opposing end to the downstream end. Reference to the radial direction or lateral direction refers to a direction that is perpendicular to the axial flow direction.

In order to generate an aerosol, the aerosol-forming substrate comprises at least one volatile compound that is intended to be vaporised/aerosolised and that may provide the user with a recreational and/or medicinal effect when inhaled. Suitable chemical and/or physiologically active volatile compounds include the group consisting of: nicotine, cocaine, caffeine, opiates and opoids, cathine and cathinone, kavalactones, mysticin, beta-carboline alkaloids, salvinorin A together with any combinations, functional equivalents to, and/or synthetic alternatives of the foregoing. The aerosol-forming substrate may comprise plant material. The plant material may comprise least one plant material selected from the list including Amaranthus dubius, Arctostaphylos uva-ursi (Bearberry), Argemone mexicana, amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), 5 Cytisus scoparius, Damiana, Entada rheedii, Eschscholzia californica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

Preferably, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above mentioned tobaccos.

Any suitable parts of the tobacco plant may be used. This includes leaves, stems, roots, bark, seeds and flowers.

The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g. slurry recon or paper recon).

The aerosol-forming substrate may comprise a gathered sheet of homogenised (e.g. paper/slurry recon) tobacco or gathered shreds/strips formed from such a sheet.

In some embodiments, the sheet used to form the aerosol-forming substrate has a grammage greater than or equal to 100 g/m², e.g. greater than or equal to 110 g/m² such as greater than or equal to 120 g/m².

The sheet may have a grammage of less than or equal to 300 g/m² e.g. less than or equal to 250 g/m2 or less than or equal to 200 g/m².

The sheet may have a grammage of between 120 and 190 g/m².

The aerosol-forming substrate may comprise at least 50 wt % plant material, e.g. at least 60 wt % plant material e.g. around 65 wt % plant material. The aerosol-forming substrate may comprise 80 wt % or less plant material e.g. 75 or 70 wt % or less plant material. The aerosol-forming substrate may comprise from 50 to 80 wt % plant material, for example from 50 to 75 wt %, from 55 to 80 wt %, from 55 to 75 wt %, from 50 to 70 wt %, from 55 to 70 wt %, from 60 to 75 wt % or from 60 to 70 wt %.

The aerosol-forming substrate may comprise one or more additives selected from humectants, flavourants, fillers, aqueous/non-aqueous solvents and binders.

Humectants are provided as vapour generators—the resulting vapour helps carry the volatile active compounds and increases visible vapour. Suitable humectants include polyhydric alcohols (e.g. propylene glycol (PG), triethylene glycol, 1,2-butane diol and vegetable glycerine (VG)) and their esters (e.g. glycerol mono-, di- or tri-acetate). They may be present in the aerosol-forming substrate in an amount between 1 and 50 wt %.

The humectant content of the aerosol-forming substrate may have a lower limit of at least 1% by weight of the plant material, such as at least 2 wt %, such as at least 5 wt %, such as at least 10 wt %, such as at least 20 wt %, such as at least 30 wt %, or such as least 40 wt %.

The humectant content of the aerosol-forming substrate may have an upper limit of at most 50% by weight of the plant material, such as at most 40 wt %, such as at most 30 wt %, or such as at most 20 wt %.

Preferably, the humectant content is 1 to 40 wt % of the aerosol-forming substrate, such as 1 to 20 wt %

Suitable binders are known in the art and may act to bind together the components forming the aerosol-forming substrate. Binders may comprise starches and/or cellulosic binders such as methyl cellulose, ethyl cellulose, hydroxypropyl cellulose, hydroxyethyl cellulose and methyl cellulose, gums such as xanthan, guar, arabic and/or locust bean gum, organic acids and their salts such as alginic acid/sodium alginate, agar and pectins.

Preferably the binder content is 5 to 10 wt % of the aerosol-forming substrate e.g. around 6 to 8 wt %.

Suitable fillers are known in the art and may act to strengthen the aerosol-forming substrate. Fillers may comprise fibrous (non-tobacco) fillers such as cellulose fibres, lignocellulose fibres (e.g. wood fibres), jute fibres and combinations thereof.

Preferably, the filler content is 5 to 10 wt % of the aerosol-forming substrate e.g. around 6 to 9 wt %.

The aerosol-forming substrate may comprise an aqueous and/or non-aqueous solvent. In some embodiments, the aerosol forming substrate has a water content of between 5 and 10 wt % e.g. between 6-9 wt % such as between 7-9 wt %.

The flavourant may be provided in solid or liquid form. It may include menthol, liquorice, chocolate, fruit flavour (including e.g. citrus, cherry etc.), vanilla, spice (e.g. ginger, cinnamon) and tobacco flavour. The flavourant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm e.g. between 6 and 9 mm or 6 and 8 mm e.g. around 7 mm. It may have an axial length of between 10 and 15 mm e.g. between 11 and 14 mm such as around 12 or 13 mm.

In some embodiments, the at least one projection is a circumferentially-extending projection around the cylindrical outer surface of the article. In some embodiments, the at least one projection is an annular projection (e.g. a collar) circumscribing the outer surface.

The article may be at least partly circumscribed by a wrapping layer e.g. a paper wrapping layer. The wrapping layer may overlie an inner foil layer or may comprise a paper/foil laminate (with the foil innermost).

In some embodiments the at least one projection is provided on the wrapping layer. In some embodiments the at least one projection is an integral part of the wrapping layer e.g. the wrapping layer may be embossed or folded (transversely) to provide the at least one projection. The at least one projection may alternatively be provided by an insert (e.g. an annular insert) provided under the wrapping layer which forces a portion of the wrapping layer radially outwards.

In some embodiments, the at least one projection is closer to the downstream axial end of the article than the upstream axial end. In this way, a greater extent of insertion of the consumable is achieved before the raised feature comes into contact with the heating device to prevent further insertion.

The article/consumable may comprise at least one filter element. There may be a terminal filter element at the downstream/mouth end of the article/consumable. There may be an upstream filter element (upstream of the downstream axial end). The upstream and terminal filter elements may be axially adjacent or may be axially spaced.

The or at least one of the filter element(s) (e.g. the terminal filter element and/or the upstream filter element) may be comprised of cellulose acetate or polypropylene tow. The or at least one of the filter element(s) (e.g. the terminal filter element and/or the upstream filter element) may be comprised of activated charcoal.

The or at least one of the filter element(s) (e.g. the terminal filter element and/or the upstream filter element) may be comprised of paper. The or at least one of the filter element(s) (e.g. the terminal filter element and/or the upstream filter element) may be comprised of extruded plant material. The or each filter element may be circumscribed with a plug wrap e.g. a paper plug wrap.

The or each filter element may have a substantially cylindrical shape with a diameter substantially matching the diameter of the aerosol-forming substrate (with or without its associated wrapping layer). The axial length of the or each filter element may be less than 20 mm, e.g. between 8 and 15 mm, for example between 9 and 13 mm e.g. between 10 and 12 mm.

The or at least one of the filter element(s) may be a solid filter element. The or at least one of the filter element(s) may be a hollow bore filter element. The or each hollow bore filter element may have a bore diameter of between 1 and 5 mm, e.g. between 2 and 4 mm or between 2 and 3 mm.

The upstream filter element may be at least partly (e.g. entirely) circumscribed by the (paper) wrapping layer.

The terminal filter element (at the downstream end of the article/consumable) may be joined to the upstream elements forming the article/consumable by a circumscribing tipping layer e.g. a tipping paper layer. The tipping paper may have an axial length longer than the axial length of the terminal filter element such that the tipping paper completely circumscribes the terminal filter element plus the wrapping layer surrounding any adjacent upstream element.

In some embodiments the at least one projection is provided on the tipping paper. In some embodiments the at least one projection is an integral part of the tipping paper e.g. the tipping paper may be embossed or folded (transversely) to provide the at least one radially-extending projection. The at least one projection may alternatively be provided by an insert (e.g. an annular insert) provided under the tipping layer which forces a portion of the tipping paper radially outwards.

The or at least one of the filter elements e.g. the terminal filter element may include a capsule e.g. a crushable capsule (crush-ball) containing a liquid flavourant e.g. any of the flavourants listed above. The capsule can be crushed by the user during smoking of the article/consumable to release the flavourant. The capsule may be located at the axial centre of the terminal filter element.

In some embodiments, the article/consumable may comprise an aerosol-cooling element which is adapted to cool the aerosol generated from the aerosol-forming substrate (by heat exchange) before being inhaled by the user.

The aerosol-cooling element will be downstream from the aerosol-forming substrate. For example, it may be between the aerosol-forming substrate and the upstream filter element and/or between the two filter elements. The aerosol cooling element may be at least partly (e.g. completely) circumscribed by the (paper) wrapping layer.

The aerosol-cooling element may be formed of a plastics material selected from the group consisting of polylactic acid (PLA), polyvinyl chloride (PVC), polyethylene (PE) and polyethylene terephthalate (PET). The aerosol-cooling element may be formed of a crimped/gathered sheet of material to form a structure having a high surface area with a plurality of longitudinal channels to maximise heat exchange and cooling of the aerosol.

The article/consumable may comprise a spacer element that defines a space or cavity or chamber between the aerosol-forming substrate and the downstream end of the article/consumable. For example, it may be provided between the aerosol-forming substrate and the upstream filter element and/or between the two filter elements. The spacer acts to allow both cooling and mixing of the aerosol. The spacer element may comprise a tubular element e.g. cardboard tube. The spacer element may be at least partly (e.g. entirely) circumscribed by the (paper) wrapping layer.

In some embodiments of the eighth mode, the consumable comprises one or more ventilation holes in the wrapping layer surrounding the spacer element. In this way, additional ventilation is provided which may enhance the user experience. As the user draws on the article/consumable, air will pass not only into the distal end of the article/consumable through the aerosol-forming substrate, but also through the one or more ventilation holes. This may provide inter alia an enhanced cooling effect on the vapour to facilitate aerosol formation, improved homogeneous mixing of the smoke/aerosol constituents and a more desirable resistance to draw (RTD). The number and size of the ventilation holes can be selected to provide a desired RTD for the article/consumable. In some embodiments, the one or more ventilation holes are located between the at least one projection and the upstream axial end of the article/consumable.

The spacer element may have an external diameter of between 5 and 10 mm e.g. between 6 and 9 mm or 6 and 8 mm e.g. around 7 mm. It may have an axial length of between 10 and 15 mm e.g. between 12 and 14 mm or 13 and 14 mm e.g. around 14 mm.

In a second aspect of the eighth mode, there is provided a smoking substitute system comprising an aerosol-forming article according to the first aspect and a device comprising a heating element.

The device may be a HNB device i.e. a device adapted to heat but not combust the aerosol-forming substrate.

The device may comprise a main body for housing the heating element. The heating element may comprise an elongated e.g. rod, tube-shaped or blade heating element. The heating element may project into or surround a cavity within the main body for receiving the article/consumable described above.

The heating device may include an annular recess proximal an opening of the cavity for engagement of the at least one projection on the article/consumable.

In this way, as the article/consumable is inserted into the device, the projection will engage within the recess, i.e. the projection/collar will “mate” with the recess. This provides the user with haptic feedback that the article/consumable is correctly inserted into the device. Furthermore, the engagement increases the resistance to movement of the article/consumable in an axial direction, both further into and back out of the cavity of the device. This provides improved control over the position of the article/consumable within the device to ensure optimal heating during use, and also prevents inadvertent removal or loss of the article/consumable from the device.

The device (e.g. the main body) may further comprise an electrical power supply e.g. a (rechargeable) battery for powering the heating element. It may further comprise a control unit to control the supply of power to the heating element.

In a third aspect of the eighth mode, there is provided a method of using a smoking substitute system according to the second aspect, the method comprising: inserting the article/consumable into the device; and heating the article/consumable using the heating element.

In some embodiments of the eighth mode, the method comprises inserting the article/consumable into a cavity within the main body and penetrating the article/consumable with the heating element upon insertion of the article/consumable. For example, the heating element may penetrate the aerosol-forming substrate in the article/consumable.

In some embodiments of the eighth mode, the method comprises inserting the article/consumable such that the at least one projection engages with the recess proximal the opening of the cavity in the device.

The skilled person will appreciate that except where mutually exclusive, a feature or parameter described in relation to any one of the above aspects of the eighth mode may be applied to any other aspect of the eighth mode. Furthermore, except where mutually exclusive, any feature or parameter described herein may be applied to any aspect and/or combined with any other feature or parameter of the eighth mode described herein.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1 shows a first embodiment of the first mode of an HNB consumable;

FIG. 2 shows a second embodiment the first mode of an HNB consumable;

FIG. 3 shows a third embodiment of the first mode of an HNB consumable;

FIG. 4 shows the first embodiment of the first mode within a device forming an HNB system;

FIG. 5 shows a first embodiment of a second mode of an HNB consumable;

FIG. 6 shows a second embodiment of the second mode of an HNB consumable;

FIG. 7 shows a third embodiment of the second mode of an HNB consumable;

FIG. 8 shows the first embodiment of the second mode within a device forming an HNB system;

FIG. 9 shows a first embodiment of a third mode of an HNB consumable;

FIG. 10 shows a second embodiment of the third mode of an HNB consumable;

FIG. 11 shows a third embodiment of the third mode of an HNB consumable;

FIG. 12 shows the first embodiment of the third mode within a device forming an HNB system;

FIG. 13 shows a first embodiment of a fourth mode of an HNB consumable;

FIG. 14 shows a second embodiment of the fourth mode of an HNB consumable;

FIG. 15 shows a third embodiment of the fourth mode of an HNB consumable;

FIG. 16 shows the first embodiment of the fourth mode within a device forming an HNB system;

FIG. 17A is an end view of a filter element of the first embodiment of the fourth mode;

FIG. 17B is a schematic view of the filter element of the first embodiment of the fourth mode and a heating element;

FIGS. 18A, 18B and 18C are end views of further embodiments of a filter element of the fourth mode.

FIG. 19 shows a cross-sectional view of a first embodiment of a fifth mode of an HNB consumable;

FIG. 20 shows a cross-sectional view of a second embodiment of the fifth mode of an HNB consumable;

FIG. 21 shows a cross-sectional view of a third embodiment of the fifth mode of an HNB consumable;

FIG. 22 shows a cross-sectional view of the first embodiment of the fifth mode within a device forming an HNB system;

FIG. 23 shows a cross-sectional view of a first embodiment of a sixth mode of an HNB consumable;

FIG. 24 shows a cross-sectional view of a second embodiment of the sixth mode of an HNB consumable;

FIG. 25 shows a cross-sectional view of a third embodiment of the sixth mode of an HNB consumable;

FIG. 26 shows cross-sectional view of the first embodiment of the sixth mode within a device forming an HNB system;

FIG. 27 shows a first embodiment of a seventh mode of an HNB consumable;

FIG. 28 shows a second embodiment of the seventh mode of an HNB consumable;

FIG. 29 shows a third embodiment of the seventh mode of an HNB consumable;

FIG. 30 shows the first embodiment of the seventh mode within a device forming an HNB system;

FIG. 31 shows an illustrative diagram of an embodiment of the manufacturing process;

FIG. 32 shows a fourth embodiment of the seventh mode of an HNB consumable;

FIG. 33 shows a fifth embodiment of the seventh mode of an HNB consumable

FIG. 34 shows a sixth embodiment of the seventh mode of an HNB consumable;

FIG. 35 shows the fourth embodiment of the seventh mode within a device forming an HNB system;

FIG. 36 shows a cross-sectional view of a first embodiment of an eighth mode of an HNB consumable;

FIG. 37 shows a cross-sectional view of a second embodiment of the eighth mode of an HNB consumable;

FIG. 38 shows a cross-sectional view of a third embodiment of the eighth mode of an HNB consumable;

FIG. 39 shows a cross-sectional view of a fourth embodiment of an eighth mode of an HNB consumable;

FIG. 40 shows a cross-sectional view of a HNB device adapted to receive a consumable according to the disclosure;

FIG. 41 shows a cross-sectional view of the first embodiment of the eighth mode within a device forming an HNB system; and

FIG. 42 shows a cross-sectional view of a fifth embodiment of the eighth mode of an HNB consumable, inserted into a HNB device forming an HNB system.

### DETAILED DESCRIPTION OF THE FIGURES

First Mode of the Disclosure: Smoking Substitute Article with Elongated Heating Element

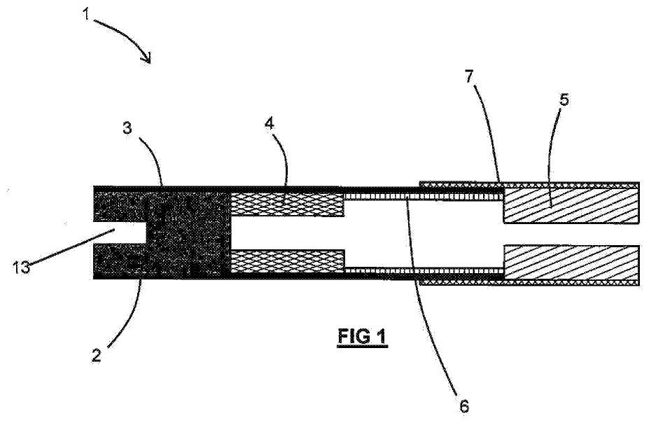

As shown in FIG. 1, the HNB consumable 1 comprises an aerosol-forming substrate 2 at the upstream end of the consumable 1.

The aerosol-forming substrate 2 comprises a rod-shaped extrudate of tobacco and is dosed with 20 wt % of a humectant such as propylene glycol (PG) or vegetable glycerine (VG).

The aerosol-forming substrate 2 is formed in a substantially cylindrical shape such that the consumable resembles a conventional cigarette. It has diameter of around 7 mm and an axial length of around 12 mm. The extrudate comprises an axially-extending recess which has its axial upstream end at the axial upstream end of the aerosol-forming substrate 2. The axially-extending recess 13 extends about a third of the length of the aerosol-forming substrate 2 and thus has an axial length of around 4 mm. It has a bore diameter of around 2 mm and a blunt downstream axial end.

The aerosol-forming substrate 2 is circumscribed by a paper wrapping layer 3.

The consumable 1 comprises an upstream filter element 4 and a downstream (terminal) filter element 5. The two filter elements 4, 5 and spaced by a cardboard spacer tube 6. Both filter elements 4, 5 are formed of cellulose acetate tow and wrapped with a respective paper plug layer (not shown).

Both filter elements have a substantially cylindrical shape. The diameter of the upstream filter 4 matches the diameter of the aerosol-forming substrate 2. The diameter of the terminal filter element 5 is slightly larger and matches the combined diameter of the aerosol-forming substrate 2 and the wrapping layer 3. The upstream filter element is slightly shorter in axial length than the terminal filter element at an axial length of 10 mm compared to 12 mm for the terminal filter element.

The cardboard tube spacer is longer than each of the two filter portions having an axial length of around 14 mm.

Each filter element 4, 5 is a hollow bore filter element with a hollow, longitudinally extending bore. The diameter of the bore in the upstream filter is slightly larger than the diameter of the bore in the terminal filter having a diameter of 3 mm compared to 2 mm for the terminal filter element.

The cardboard spacer tube 6 and the upstream filter portion 4 are circumscribed by the wrapping layer 3.

The terminal filter element 5 is joined to the upstream elements forming the consumable by a circumscribing paper tipping layer 7. The tipping layer 7 encircles the terminal filter portion and has an axial length of around 20 mm such that it overlays a portion of the cardboard tube spacer 6.

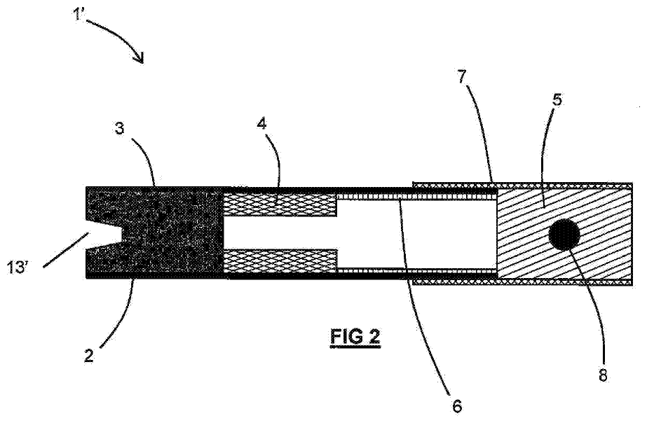

FIG. 2 shows a second embodiment of a consumable 1′ which is the same as that shown in FIG. 1 except that the terminal filter element 5 is a solid filter element and comprises a crushable capsule 8 (crush-ball) having a shell wall containing a liquid menthol or cherry or vanilla flavourant. The capsule 8 is spherical and has a diameter of 3.5 mm. It is positioned within the axial centre of the terminal filter portion 5. The capsule 8 may be omitted from the FIG. 2 embodiment and may be included in the FIG. 1 embodiment. The axially-extending recess 13′ in the FIG. 2 embodiment is tapered from a diameter of around 2 mm to a diameter of around 1 mm.

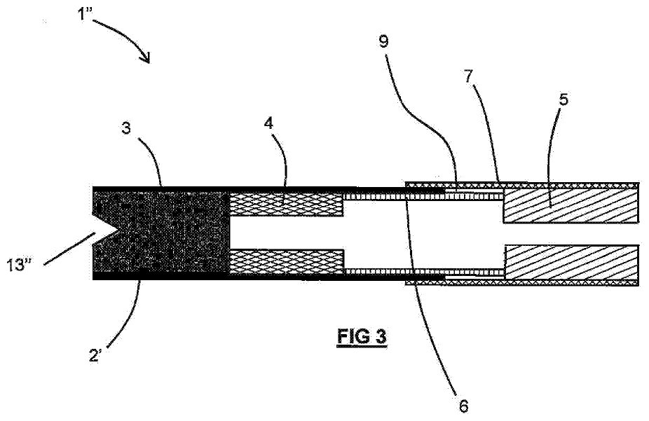

FIG. 3 shows a third embodiment of a consumable 1″ which is the same as the first embodiment except that the wrapping layer 3 does not completely circumscribe the cardboard spacer tube 6 such that there is an annular gap 9 between the tipping layer 7 and the cardboard spacer tube 6 downstream of the end of the wrapping layer 3. Furthermore, the axially extending recess 13″ tapers from a diameter of around 2 mm to a point. Finally, the extruded rod of tobacco is replaced with an aerosol-forming substrate 2′ comprising 65 wt % tobacco which is provided in the form of gathered shreds produced from a sheet of slurry/paper recon tobacco. The tobacco is dosed with 20 wt % of a humectant such as propylene glycol (PG) or vegetable glycerine (VG) and has a moisture content of between 7-9 wt %. The aerosol-forming substrate further comprises cellulose pulp filler and guar gum binder.

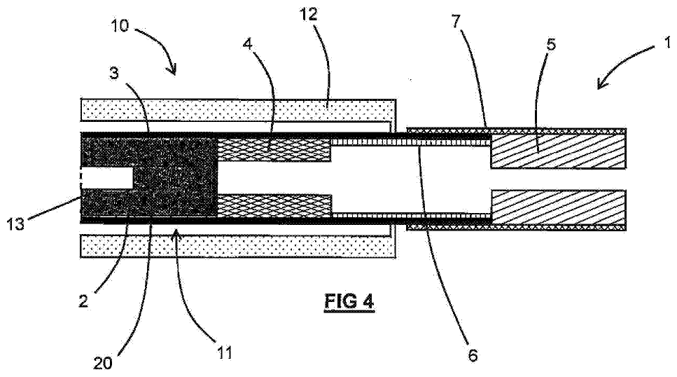

FIG. 4 shows the first embodiment inserted into an HNB device 10 comprising a rod-shaped heating element (not shown). The heating element projects into a cavity 11 within the main body 12 of the device.

The consumable 1 is inserted into the cavity 11 of the main body 12 of the device 10 such that the heating rod 20 is received in the axially-extending recess 13 of the aerosol-forming substrate 2. Heating of the reconstituted tobacco in the aerosol-forming substrate 2 is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter portion 5.

As the vapour cools within the upstream filter element 4 and the cardboard spacer tube 6, it condenses to form an aerosol containing the volatile compounds for inhalation by the user.

Second Mode of the Disclosure: Cooling Element Formed of Plant Material

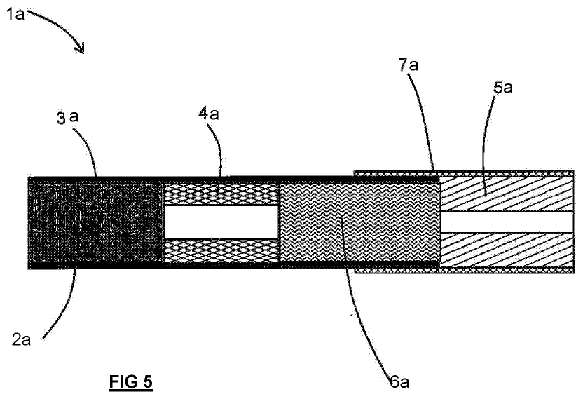

As shown in FIG. 5, the HNB consumable 1a comprises an aerosol-forming substrate 2a at the upstream end of the consumable 1a.

The aerosol-forming substrate 2a comprises 65 wt % tobacco which is provided in the form of gathered shreds produced from a sheet of slurry/paper recon tobacco. The tobacco is dosed with 20 wt % of a humectant such as propylene glycol (PG) or vegetable glycerine (VG) and has a moisture content of between 7-9 wt %. The aerosol-forming substrate 2a further comprises cellulose pulp filler and guar gum binder.

The aerosol-forming substrate 2a is formed in a substantially cylindrical shape such that the consumable resembles a conventional cigarette. It has diameter of around 7 mm and an axial length of around 12 mm.

The aerosol-forming substrate 2a is circumscribed by a paper wrapping layer 3a.

The consumable 1a comprises an upstream filter element 4a and a downstream (terminal) filter element 5a. The two filter elements 4a, 5a and spaced by an aerosol-cooling element 6a. Both filter elements 4a, 5a are formed of cellulose acetate tow and wrapped with a respective paper plug layer (not shown).

Both filter elements 4a, 5a have a substantially cylindrical shape. The diameter of the upstream filter 4a matches the diameter of the aerosol-forming substrate 2a. The diameter of the terminal filter element 5a is slightly larger and matches the combined diameter of the aerosol-forming substrate 2a and the wrapping layer 3a. The upstream filter element is slightly shorter in axial length than the terminal filter element at an axial length of 10 mm compared to 12 mm for the terminal filter element.

Each filter element 4a, 5a is a hollow bore filter element with a hollow, longitudinally extending bore. The diameter of the bore in the upstream filter is slightly larger than the diameter of the bore in the terminal filter having a diameter of 3 mm compared to 2 mm for the terminal filter element.

The aerosol-cooling element 6a has a substantially cylindrical shape and has a diameter that matches that of the upstream filter element 4a. It is formed of a gathered and crimped sheet of slurry or paper recon tobacco and has a large surface area so that aerosol passing downstream through it is cooled by heat exchange.

The aerosol-cooling element 6a and the upstream filter element 4a are circumscribed by the wrapping layer 3a.

The terminal filter element 5a is joined to the upstream elements forming the consumable by a circumscribing paper tipping layer 7a. The tipping layer 7a encircles the terminal filter portion and has an axial length of around 20 mm such that it overlays a portion of the aerosol-cooling element 6a.

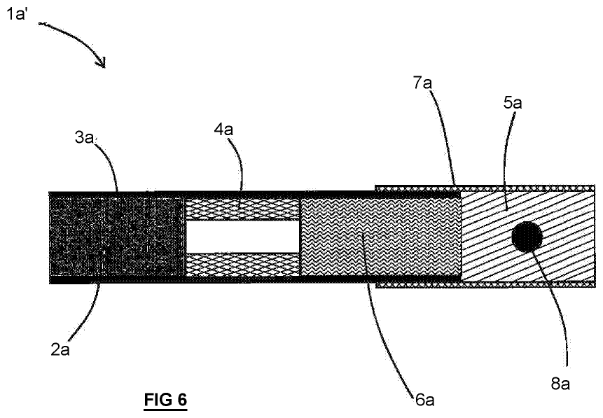

FIG. 6 shows a second embodiment of a consumable 1a′ which is the same as that shown in FIG. 5 except that the aerosol-cooling element 6a′ is formed of granules/chips/pellets of extruded tobacco.

The terminal filter element 5a is a solid filter element and comprises a crushable capsule 8a (crush-ball) having a shell wall containing a liquid menthol or cherry or vanilla flavourant. The capsule 8a is spherical and has a diameter of 3.5 mm. It is positioned within the axial centre of the terminal filter portion 5a. In other embodiments (not shown), the capsule 8a can be omitted.

Furthermore, the capsule 8a could be included in a solid terminal filter portion 5a of the FIG. 5 embodiment.

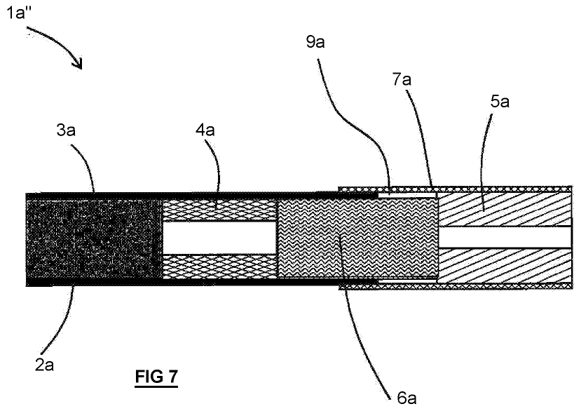

FIG. 7 shows a third embodiment of a consumable 1a″ which is the same as the first embodiment except that the wrapping layer 3a does not completely circumscribe the aerosol-cooling element 6a such that there is an annular gap 9a between the tipping layer 7a and the aerosol-cooling element 6a downstream of the end of the wrapping layer 3a. In the FIG. 7 embodiment, the recon tobacco aerosol-cooling element 6a could be replaced with the extruded tobacco aerosol-cooling element 6a′ of the FIG. 6 embodiment and/or the crushable capsule 8a of the FIG. 6 may be included in the terminal filter portion 5a.

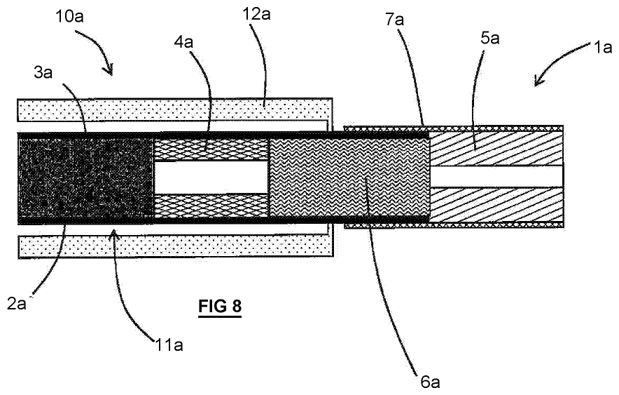

FIG. 8 shows the first embodiment inserted into an HNB device 10a comprising a rod-shaped heating element (not shown). The heating element projects into a cavity 11a within the main body 12a of the device.

The consumable 1a is inserted into the cavity 11a of the main body 12a of the device 10a such that the heating rod penetrates the aerosol-forming substrate 2a. Heating of the reconstituted tobacco in the aerosol-forming substrate 2a is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter portion 5a. The crushable capsule 8a can be ruptured by pressure to modify the flavour and/or amount of visible vapour during smoking of the consumable.

As the vapour cools within the upstream filter element 4a and the aerosol-cooling element 6a, it condenses to form an aerosol containing the volatile compounds for inhalation by the user.

Third Mode: Extruded Plant Material

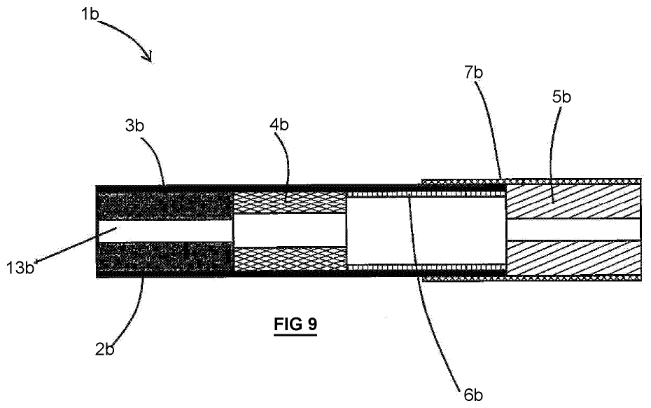

As shown in FIG. 9, the HNB consumable 1b comprises an aerosol-forming substrate 2b at the upstream end of the consumable 1b.

The aerosol-forming substrate 2b comprises a rod-shaped extrudate of tobacco and is dosed with 20 wt % of a humectant such as propylene glycol (PG) or vegetable glycerine (VG).

The aerosol-forming substrate 2b is formed in a substantially cylindrical shape such that the consumable resembles a conventional cigarette. It has diameter of around 7 mm and an axial length of around 12 mm. The extrudate comprises an axial bore 13b which has its axial upstream end at the axial upstream end of the aerosol-forming substrate 2b. The axial bore 13b extends the entire length of the extrudate and thus has an axial length of 12 mm. It has a bore diameter of around 2 mm.

The aerosol-forming substrate 2b is circumscribed by a paper wrapping layer 3b.

The consumable 1b comprises an upstream filter element 4b and a downstream (terminal) filter element 5b. The two filter elements 4b, 5b and spaced by a cardboard spacer tube 6b. Both filter elements 4b, 5b are formed of cellulose acetate tow and wrapped with a respective paper plug layer (not shown).

Both filter elements 4b, 5b have a substantially cylindrical shape. The diameter of the upstream filter element 4b matches the diameter of the aerosol-forming substrate 2b. The diameter of the terminal filter element 5b is slightly larger and matches the combined diameter of the aerosol-forming substrate 2b and the wrapping layer 3b. The upstream filter element 4b is slightly shorter in axial length than the terminal filter element 5b at an axial length of 10 mm compared to 12 mm for the terminal filter element 5b.

The cardboard tube spacer is longer than each of the two filter elements having an axial length of around 14 mm.

Each filter element 4b, 5b is a hollow bore filter element with a hollow, longitudinally extending bore. The diameter of the bore in the upstream filter element 4b is slightly larger than the diameter of the bore in the terminal filter element 5b having a diameter of 3 mm compared to 2 mm for the terminal filter element 5b.

The cardboard spacer tube 6b and the upstream filter element 4b are circumscribed by the wrapping layer 3b.

The terminal filter element 5b is joined to the upstream elements forming the consumable by a circumscribing paper tipping layer 7b. The tipping layer 7b encircles the terminal filter portion and has an axial length of around 20 mm such that it overlays a portion of the cardboard tube spacer 6b.

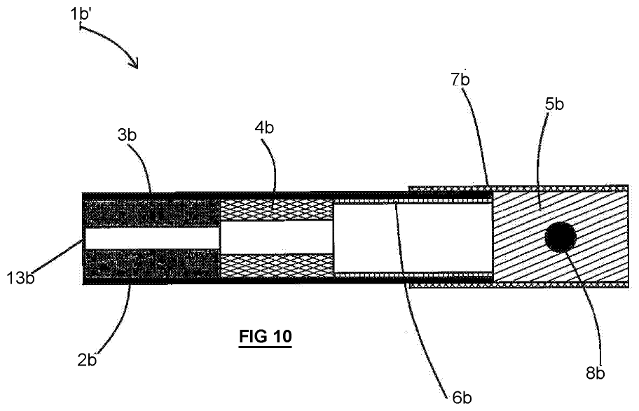

FIG. 10 shows a second embodiment of a consumable 1a′ which is the same as that shown in FIG. 9 except that the terminal filter element 5a is a solid filter element and comprises a crushable capsule 8a (crush-ball) having a shell wall containing a liquid menthol or cherry or vanilla flavourant. The capsule 8a is spherical and has a diameter of 3.5 mm. It is positioned within the axial centre of the terminal filter portion 5b. The capsule 8b may be omitted from the FIG. 10 embodiment and may be included in the FIG. 9 embodiment.

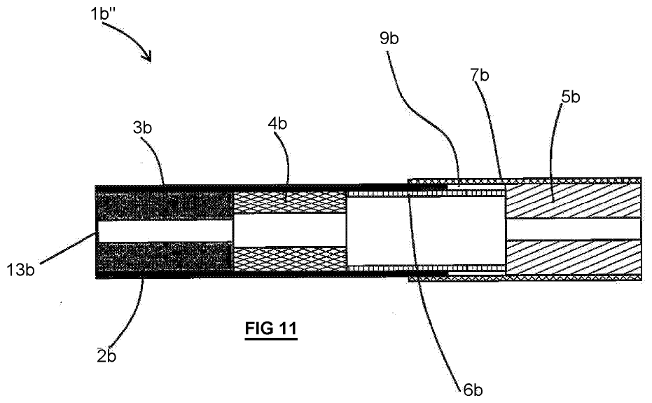

FIG. 11 shows a third embodiment of a consumable 1b″ which is the same as the first embodiment of the third mode except that the wrapping layer 3b does not completely circumscribe the cardboard spacer tube 6b such that there is an annular gap 9b between the tipping layer 7b and the cardboard spacer tube 6b downstream of the end of the wrapping layer 3b.

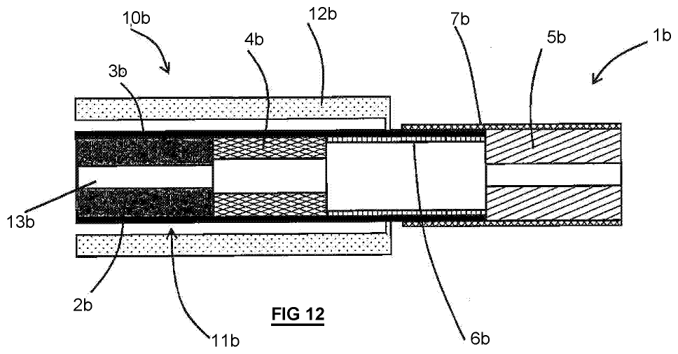

FIG. 12 shows the first embodiment of the third mode inserted into an HNB device 10b comprising a rod-shaped heating element (not shown). The heating element projects into a cavity 11b within the main body 12b of the device.

The consumable 1b is inserted into the cavity 11b of the main body 12b of the device 10b such that the heating rod is received in the axial bore 13b of the aerosol-forming substrate 2b. Heating of the reconstituted tobacco in the aerosol-forming substrate 2b is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter portion 5b.

As the vapour cools within the upstream filter element 4b and the cardboard spacer tube 6b, it condenses to form an aerosol containing the volatile compounds for inhalation by the user.

Fourth Mode: Filter Having One or More Bores

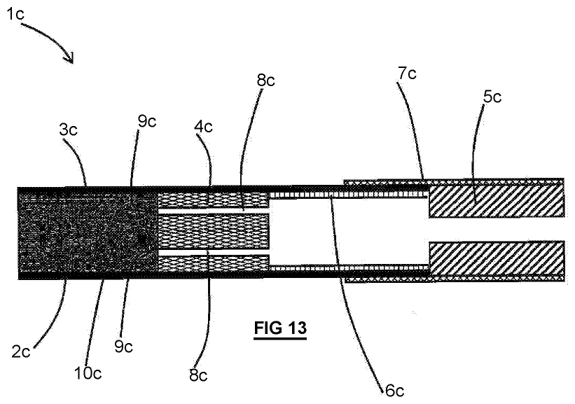

As shown in FIG. 13, the HNB consumable 1c comprises an aerosol-forming substrate 2c at the upstream end of the consumable 1c.

The aerosol-forming substrate 2c comprises reconstituted tobacco which includes nicotine as a volatile compound.

The aerosol-forming substrate 2c is dosed with 20 wt % of a humectant such as propylene glycol (PG) or vegetable glycerine (VG).

The aerosol-forming substrate 2c is formed in a substantially cylindrical shape such that the consumable resembles a conventional cigarette. It has diameter of around 7 mm and an axial length of around 12 mm.

The aerosol-forming substrate 2c is circumscribed by a paper wrapping layer 3c.

The consumable 1c comprises an upstream filter element 4c and a downstream (terminal) filter element 5c. The two filter elements 4c, 5c and spaced by a cardboard spacer tube 6c. Both filter elements 4c, 5c are formed of cellulose acetate tow and wrapped with a respective paper plug layer (not shown).

Both filter elements have a substantially cylindrical shape. The diameter of the upstream filter 4c matches the diameter of the aerosol-forming substrate 2c. The diameter of the terminal filter element 5c is slightly larger and matches the combined diameter of the aerosol-forming substrate 2c and the wrapping layer 3c. The upstream filter element 4c is slightly shorter in axial length than the terminal filter element 5c at an axial length of 10 mm compared to 12 mm for the terminal filter element 5c.

The cardboard tube spacer 6c is longer than each of the two filter elements having an axial length of around 14 mm.

The terminal filter element 5c is a hollow bore filter element with a hollow, longitudinally extending bore. The diameter of the bore in the terminal filter is 2 mm.

The upstream filter element 4c comprises a plurality of bores 8c extending longitudinally through the upstream filter element 4c. Although not apparent from FIG. 13, the upstream filter element 4c includes four bores 8c with respective openings 9c in an upstream side 10c facing the aerosol-forming substrate 2c. The openings are spaced radially from a central longitudinal axis of the upstream filter element 4c (and from central longitudinal axis of the substrate 2c).

The cardboard spacer tube 6c and the upstream filter portion 4c are circumscribed by the wrapping layer 3c.

The terminal filter element 5c is joined to the upstream elements forming the consumable by a circumscribing paper tipping layer 7c. The tipping layer 7c encircles the terminal filter element 5c and has an axial length of around 20 mm such that it overlays a portion of the cardboard tube spacer 6c.

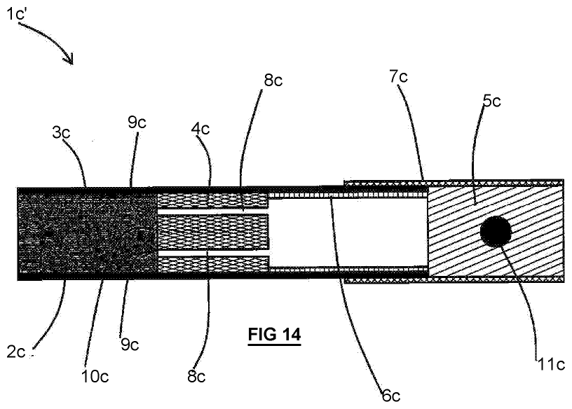

FIG. 14 shows a second embodiment of a consumable 1c′ which is the same as that shown in FIG. 13 except that the terminal filter element 5c is a solid filter element and comprises a crushable capsule 11c (crush-ball) having a shell wall containing a liquid menthol or cherry or vanilla flavourant. The capsule 11c is spherical and has a diameter of 3.5 mm. It is positioned within the axial centre of the terminal filter portion 5c.

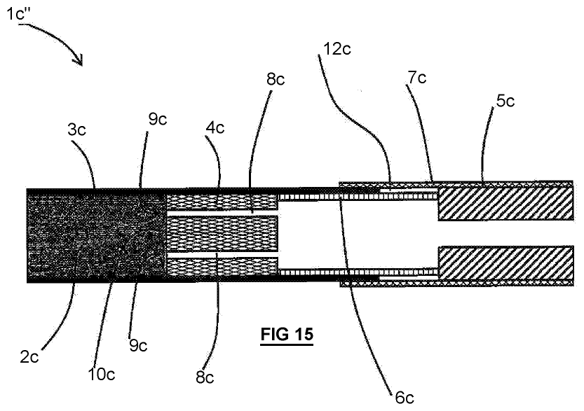

FIG. 15 shows a third embodiment of a consumable 1c″ which is the same as the first embodiment of the fourth mode except that the wrapping layer 3c does not completely circumscribe the cardboard spacer tube 6c such that there is an annular gap 12c between the tipping layer 7c and the cardboard spacer tube 6c downstream of the end of the wrapping layer 3c.

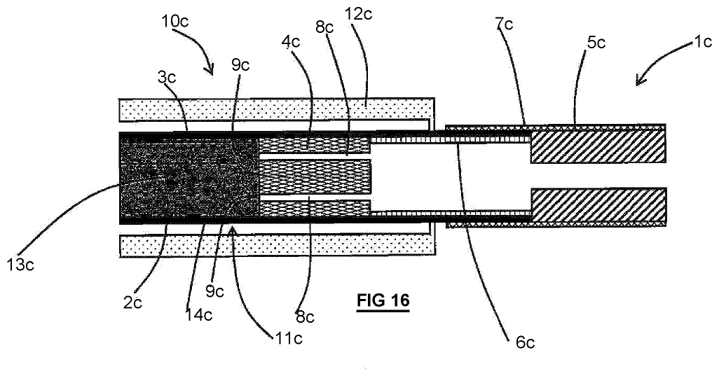

FIG. 16 shows the first embodiment inserted into an HNB device 10c comprising a rod-shaped heating element 13c (shown in dashed lines). The heating element 13c projects into a cavity 11c within the main body 12c of the device.

The consumable 1c is inserted into the cavity 11c of the main body 12c of the device 10c such that the heating element 13c penetrates the aerosol-forming substrate 2c along a longitudinal axis of the substrate 2c. Heating of the reconstituted tobacco in the aerosol-forming substrate 2c is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter element 5c.

As the vapour cools within the upstream filter element 4 and the cardboard spacer tube 6c, it condenses to form an aerosol containing the volatile compounds for inhalation by the user.

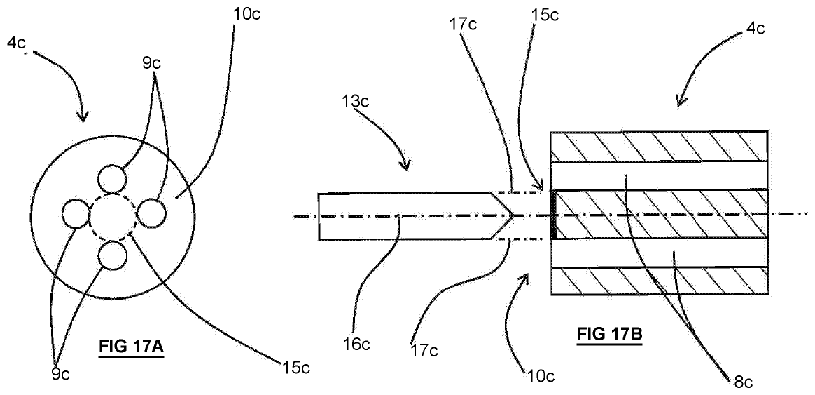

As is apparent from FIG. 16, the bores 8c of the upstream filter element 4c are arranged such their respective openings 9c are non-coincident with the longitudinal axis (along which the heating element 13c is received in the substrate 2c). That is, the openings 9c are generally unaligned with the heating element 13c along the longitudinal axis. This is more apparent from a review of FIGS. 17A and 17B.

FIG. 17A shows a front view of the upstream filter element 4c as described above (and shown in the previous figures). As is apparent from this figure, the upstream filter element 4c comprises four bores with four respective openings 9c (i.e. at the upstream axial end 14c of the upstream filter element 4c). The upstream axial end 14c of the upstream filter element 4c further comprises a projected region 15c (shown in dashed lines). The projected region 15c is centrally located on the upstream side 10c and, in the illustrated embodiment, has a diameter of 2 mm. The openings 9c are located so as to be contiguous with the periphery of the projected region 15c (i.e. the peripheral edges of the openings 9c are adjacent the periphery of the projected region 15c). The openings 9c are also spaced evenly about the projected region 15c. The projected region 15c is defined by the shape and location of a heating element (i.e. received by an aerosol-forming article of which the upstream filter 4c forms part of). This is better described with reference to FIG. 17B.

FIG. 17B is a schematic side view of the heating element 13c and upstream filter element 4c. The heating element 13c is elongate and extends along a longitudinal axis 16c. A transverse profile of the heating element 13c is circular and has the same diameter (i.e. 2 mm) as the projected region 15c of the upstream filter element 4c. The projected region 15c is defined by a projection (see projection lines 17c) of the circular transverse profile of the heating element 13c onto the upstream axial end 14c of the upstream filter element 4c.

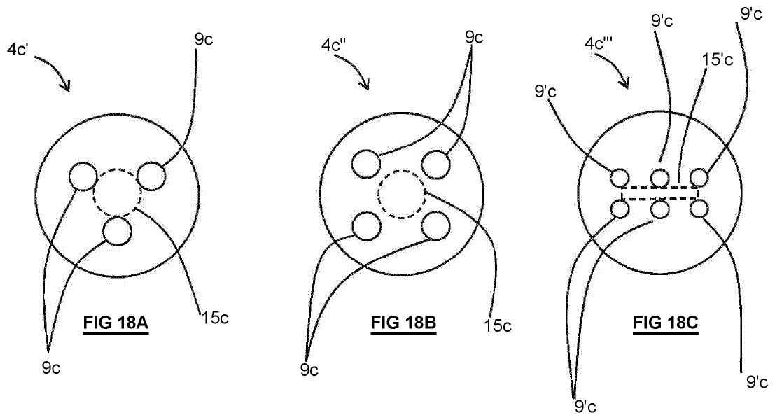

FIGS. 18A, 18B and 18C are front views of further upstream filter element embodiments 4c′, 4c″, 4c′″ that have different arrangements of bores 9c, and thus bore openings 10c.

The upstream filter element 4c′ of FIG. 18A shows a similar arrangement to that previously described, except that the upstream filter element 4c′ comprises three openings 9c (to three respective bores) rather than four openings. The three openings 9c are arranged so as to be contiguous with a circular projected region 15c of the upstream filter element 4c′, and are spaced evenly about the projected region 14c.

FIG. 18B depicts an upstream filter element 4c″ comprising four openings 9c, but rather than being contiguous with a circular projected region 15c, they are spaced from the projected region 15c. The spacing of each opening 9c from the projected region 15c is such that the opening 9c is closer to the projected region 15c than a periphery of the upstream filter 4c.

FIG. 18B depicts an upstream filter element 4c″ comprising a projected region 15c′ that has a rectangular shape. In this case, the filter element 4c″ is for use with a heating element having a rectangular transverse profile, such as a blade heating element. The upstream filter element 4c′″ comprises six openings 9c′ that are located so as to be contiguous with a periphery of the projected region 15c′. Each of the six openings 9c′ is circular, but smaller than the openings 9c shown in FIGS. 18A and 18B.

Fifth Mode of the Disclosure: Disposable in a More Environmentally Friendly Manner

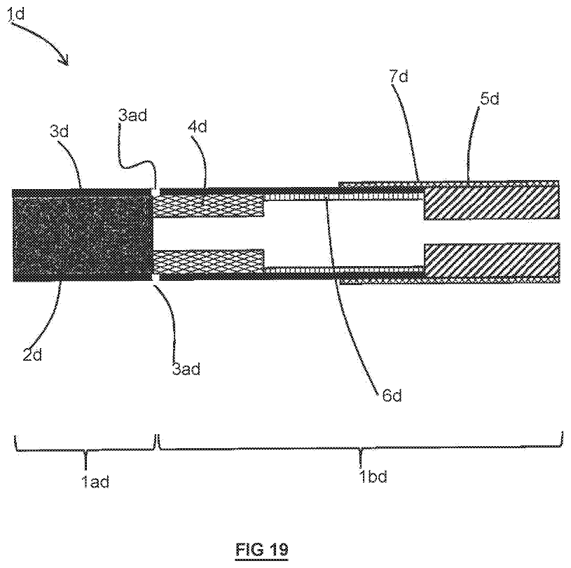

As shown in FIG. 19, the HNB consumable 1d comprises an aerosol-forming substrate 2d at the upstream end of the consumable 1d.

The aerosol-forming substrate comprises reconstituted tobacco which includes nicotine as a volatile compound.

The aerosol-forming substrate 2d comprises 65 wt % tobacco which is provided in the form of gathered shreds produced from a sheet of slurry/paper recon tobacco. The tobacco is dosed with 20 wt % of a humectant such as propylene glycol (PG) or vegetable glycerine (VG) and has a moisture content of between 7-9 wt %. The aerosol-forming substrate further comprises cellulose pulp filler and guar gum binder.

The aerosol-forming substrate 2d is formed in a substantially cylindrical shape such that the consumable resembles a conventional cigarette. It has diameter of around 7 mm and an axial length of around 12 mm.

The aerosol-forming substrate 2d is circumscribed by a paper wrapping layer 3d. The aerosol-forming substrate 2d is adapted to be heated by an external heat source as explained in more detail below.

The consumable 1d comprises an upstream filter element 4d and a downstream (terminal) filter element 5d. The two filter elements 4d, 5d and spaced by a cardboard tube spacer 6d. Both filter elements 4d, 5d are formed of cellulose acetate tow and wrapped with a respective paper plug layer (not shown).

Both filter elements 4d, 5d have a substantially cylindrical shape. The diameter of the upstream filter 4d matches the diameter of the aerosol-forming substrate 2d. The diameter of the terminal filter element 5d is slightly larger and matches the combined diameter of the aerosol-forming substrate 2d and the wrapping layer 3d. The upstream filter element is slightly shorter in axial length than the terminal filter element at an axial length of 10 mm compared to 12 mm for the terminal filter element.

The cardboard tube spacer is longer than each of the two filter elements having an axial length of around 14 mm.

Each filter element 4d, 5d is a hollow bore filter element with a hollow, longitudinally extending bore. The diameter of the bore in the upstream filter is slightly larger than the diameter of the bore in the terminal filter having a diameter of 3 mm compared to 2 mm for the terminal filter element.

The cardboard tube spacer 6d and the upstream filter element 4d are circumscribed by the wrapping layer 3d. The terminal filter element 5d is joined to the upstream elements forming the consumable by a circumscribing paper tipping layer 7d. The tipping layer 7d encircles the terminal filter element 5d and has an axial length of around 20 mm such that it overlays a portion of the cardboard tube spacer 6d.

The paper wrapping layer 3d includes an annular region of weakness in the form of an annular array of perforations 3ad through the wrapping layer at an axial position adjacent to the junction between the aerosol-forming substrate 2d and the upstream filter element 4d. The array of perforations 3ad circumscribes the consumable 1d. By breaking the consumable in two along the array of perforations 3ad the consumable is separable into a first section lad and a second section 1bd. The first section lad contains the aerosol-forming substrate 2d. The second section 1bd contains the two filter elements 4d, 5d and the cardboard tube spacer 6d.

After separation of the two sections the aerosol-forming substrate 2d can be disposed of, for example into an ashtray, and the cardboard tube spacer 6d and each filter element 4d, 5d can be separately disposed of, for example by recycling.

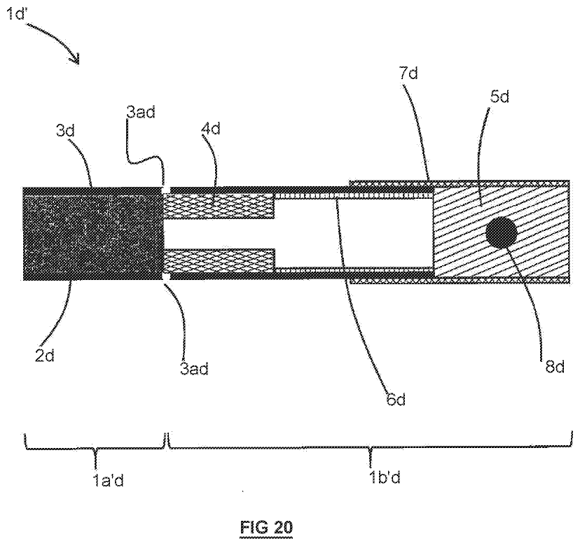

FIG. 20 shows a second embodiment of a consumable 1d′ which is the same as that shown in FIG. 19 except that the terminal filter element 5d is a solid filter element and comprises a crushable capsule 8d (crush-ball) having a shell wall containing a liquid menthol or cherry or vanilla flavourant. The capsule 8d is spherical and has a diameter of 3.5 mm. It is positioned within the axial centre of the terminal filter element 5.

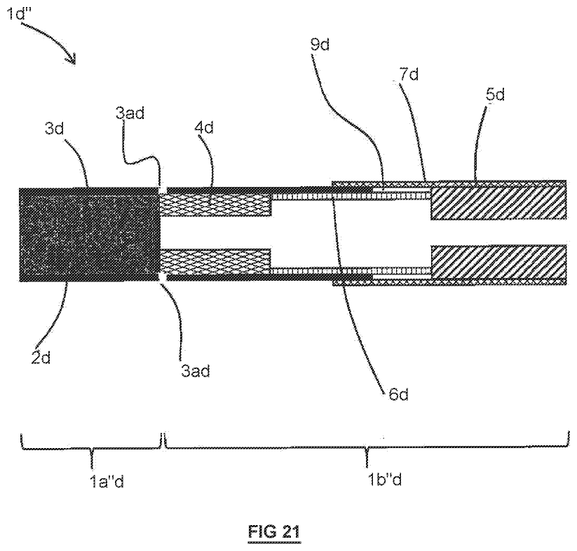

FIG. 21 shows a third embodiment of a consumable 1a″ which is the same as the first embodiment of the fifth mode except that the wrapping layer 3d does not completely circumscribe the cardboard tube spacer 6d such that there is an annular gap 9d between the tipping layer 7d and the cardboard tube spacer 6d downstream of the end of the wrapping layer 3d.

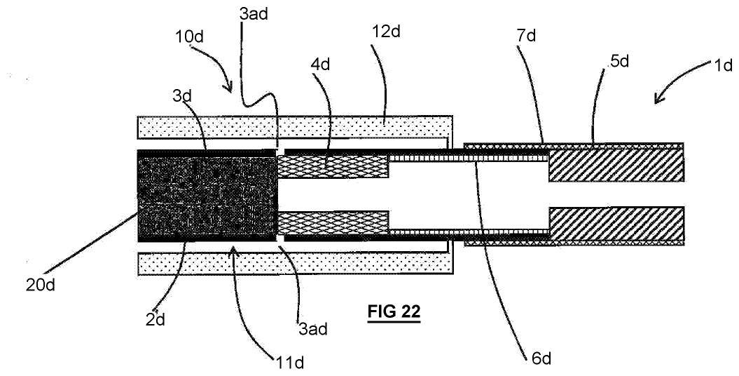

FIG. 22 shows the first embodiment of the fifth mode inserted into an HNB device 10d comprising a rod-shaped heating element 20d. The heating element 20d projects into a cavity 11d within the main body 12d of the device.

The consumable 1d is inserted into the cavity 11d of the main body 12d of the device 10d such that the heating rod 20d penetrates the aerosol-forming substrate 2d. Heating of the reconstituted tobacco in the aerosol-forming substrate 2d is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter element 5d.

As the vapour cools within the upstream filter element 4d and the cardboard tube spacer 6d, it condenses to form an aerosol containing the volatile compounds for inhalation by the user.

Sixth Mode of the Disclosure: Biodegradable Materials

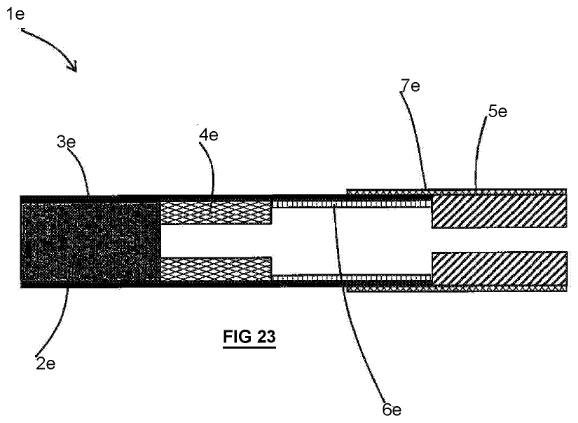

As shown in FIG. 23, the HNB consumable 1e comprises an aerosol-forming substrate 2e at the upstream end of the consumable 1e.

The aerosol-forming substrate 2e comprises reconstituted tobacco which includes nicotine as a volatile compound. The aerosol-forming substrate 2e is fully biodegradable.

The aerosol-forming substrate 2e comprises 65 wt % tobacco which is provided in the form of gathered shreds produced from a sheet of slurry/paper recon tobacco. The tobacco is dosed with 20 wt % of a humectant such as propylene glycol (PG) or vegetable glycerine (VG) and has a moisture content of between 7-9 wt %. The aerosol-forming substrate further comprises cellulose pulp filler and guar gum binder.

The aerosol-forming substrate 2e is formed in a substantially cylindrical shape such that the consumable resembles a conventional cigarette. It has diameter of around 7 mm and an axial length of around 12 mm.

The aerosol-forming substrate 2e is circumscribed by a paper wrapping layer 3e. The paper of the wrapping layer 3e does not contain any inorganic filler materials and is biodegradable. The paper wrapping layer may be a tobacco-based paper layer.

The consumable 1e comprises an upstream filter element 4e and a downstream (terminal) filter element 5e.

The two filter elements 4e, 5e and spaced by a biodegradable cardboard tube spacer 6e. Both filter elements 4e, 5e are formed of biodegradable paper and wrapped with a respective biodegradable paper plug layer (not shown).

Both filter elements have a substantially cylindrical shape. The diameter of the upstream filter 4e matches the diameter of the aerosol-forming substrate 2e. The diameter of the terminal filter element 5e is slightly larger and matches the combined diameter of the aerosol-forming substrate 2e and the wrapping layer 3e. The upstream filter element is slightly shorter in axial length than the terminal filter element at an axial length of 10 mm compared to 12 mm for the terminal filter element.

The biodegradable cardboard tube spacer is longer than each of the two filter elements having an axial length of around 14 mm. In an alternative embodiment the biodegradable spacer element 6e is made from an extruded tube of biodegradable polylactic acid (PLA).

Each filter element 4e, 5e is a hollow bore filter element with a hollow, longitudinally extending bore. The diameter of the bore in the upstream filter element 4 is slightly larger than the diameter of the bore in the terminal filter element 5e having a diameter of 3 mm compared to 2 mm for the terminal filter element 5e.

The cardboard tube spacer 6e and the upstream filter element 4e are circumscribed by the wrapping layer 3e.

The terminal filter element 5e is joined to the upstream elements forming the consumable by a circumscribing paper tipping layer 7e which is made from biodegradable paper. The tipping layer 7e encircles the terminal filter element 5e and has an axial length of around 20 mm such that it overlays a portion of the cardboard tube spacer 6e.

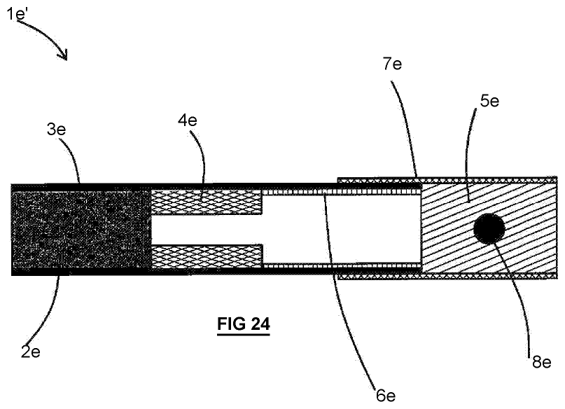

FIG. 24 shows a second embodiment of a consumable 1e′ which is the same as that shown in FIG. 23 except that the terminal filter element 5e is a solid filter element and comprises a crushable capsule 8e (crush-ball) having a shell wall made from biodegradable polymer material such as PLA and containing a liquid menthol or cherry or vanilla flavourant. The capsule 8e is spherical and has a diameter of 3.5 mm. It is positioned within the axial centre of the terminal filter element 5e.

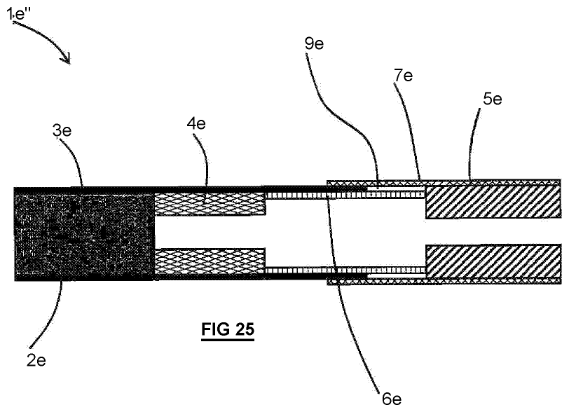

FIG. 25 shows a third embodiment of a consumable 1e″ which is the same as the first embodiment of the sixth mode except that the wrapping layer 3e does not completely circumscribe the cardboard tube spacer 6e such that there is an annular gap 9e between the tipping layer 7e and the cardboard tube spacer 6e downstream of the end of the wrapping layer 3e.

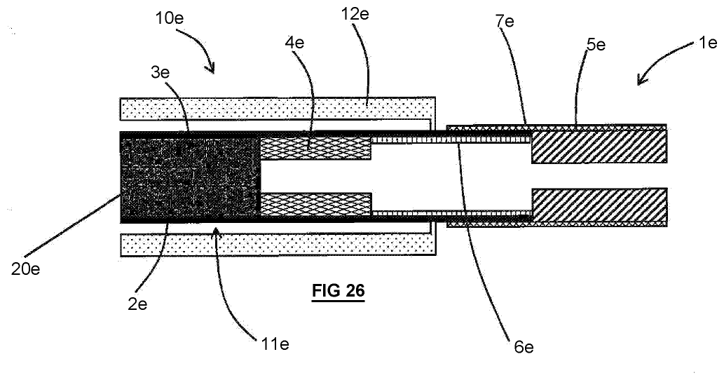

FIG. 26 shows the first embodiment of the sixth mode inserted into an HNB device 10e comprising a rod-shaped heating element 20e. The heating element 20e projects into a cavity 11e within the main body 12e of the device.

The consumable 1e is inserted into the cavity 11e of the main body 12e of the device 10e such that the heating rod 20e penetrates the aerosol-forming substrate 2e. Heating of the reconstituted tobacco in the aerosol-forming substrate 2e is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter element 5e.

As the vapour cools within the upstream filter element 4e and the cardboard tube spacer 6e, it condenses to form an aerosol containing the volatile compounds for inhalation by the user.

After use of the consumable, the user removes the consumable from the device 10e and may dispose of the consumable at a composting facility.

Seventh Mode of the Disclosure: Aerosol-Forming Article Having an Improved Cooling Element

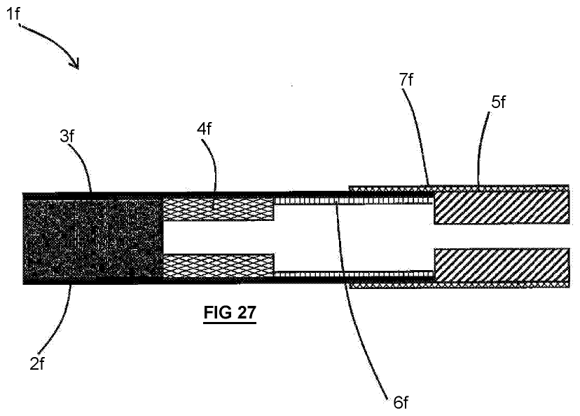

As shown in FIG. 27, the HNB consumable 1f comprises an aerosol-forming substrate 2f at the upstream end of the consumable 1f.

The aerosol-forming substrate 2f comprises reconstituted tobacco which includes nicotine as a volatile compound and which is dosed with 2 to 15 wt % of a nicotine salt selected from nicotine hydrochloride, nicotine dihydrochloride, nicotine monotartrate, nicotine bitartrate, nicotine bitartrate dihydrate, nicotine benzoate, nicotine fumarate, nictotine orotate, nicotine sulphate, nicotine zinc chloride monohydrate, nicotine lactate and nicotine salicylate, and combinations thereof.

The aerosol-forming substrate 2f comprises 65 wt % tobacco which is provided in the form of gathered shreds produced from a sheet of slurry-type reconstituted tobacco. The tobacco is dosed with 20 wt % of a humectant such as propylene glycol (PG) or vegetable glycerine (VG) and has a moisture content of between 7-9 wt %. The aerosol-forming substrate 2f further comprises cellulose pulp filler and guar gum binder.

The aerosol-forming substrate 2f is formed in a substantially cylindrical shape such that the consumable resembles a conventional cigarette. It has diameter of around 7 mm and an axial length of around 12 mm.

The aerosol-forming substrate 2f is circumscribed by a paper wrapping layer 3f.

The consumable 1f comprises an upstream filter element 4f and a downstream (terminal) filter element 5f. The two filter elements 4f, 5f are spaced by a cardboard tube spacer 6f. Both filter elements 4f, 5f are formed of cellulose acetate tow and wrapped with a respective paper plug layer (not shown).

Both filter elements 4f, 5f have a substantially cylindrical shape. The diameter of the upstream filter 4f matches the diameter of the aerosol-forming substrate 2f. The diameter of the terminal filter element 5f is slightly larger and matches the combined diameter of the aerosol-forming substrate 2f and the wrapping layer 3f. The upstream filter element 4f is slightly shorter in axial length than the terminal filter element 5f at an axial length of 10 mm compared to 12 mm for the terminal filter element 5f.

The cardboard tube spacer 6f is longer than each of the two filter portions having an axial length of around 14 mm.

Each filter element 4f, 5f is a hollow bore filter element with a hollow, longitudinally extending bore. The diameter of the bore in the upstream filter 4f is slightly larger than the diameter of the bore in the terminal filter element 5f, having a diameter of 3 mm compared to 2 mm for the terminal filter element 5f.

The cardboard tube spacer 6f and the upstream filter portion 4f are circumscribed by the wrapping layer 3f.

The terminal filter element 5f is joined to the upstream elements forming the consumable by a circumscribing paper tipping layer 7f. The tipping layer 7f encircles the terminal filter portion and has an axial length of around 20 mm such that it overlays a portion of the cardboard tube spacer 6f.

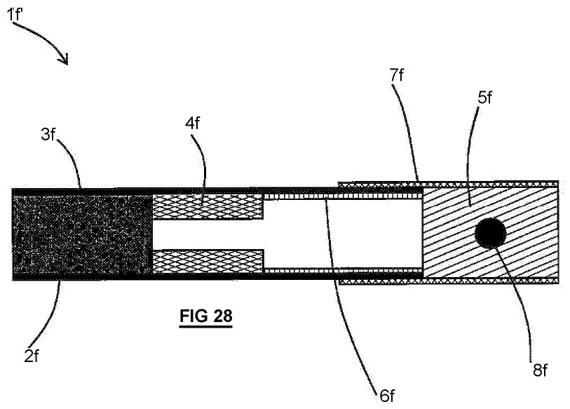

FIG. 28 shows a second embodiment of a consumable 1f which is the same as that shown in FIG. 27 except that the terminal filter element 5f comprises a crushable capsule 8f (crush-ball) containing a liquid menthol or cherry or vanilla flavourant. The capsule 8f is spherical and has a diameter of 3.5 mm. It is positioned within the axial centre of the terminal filter portion 5f.

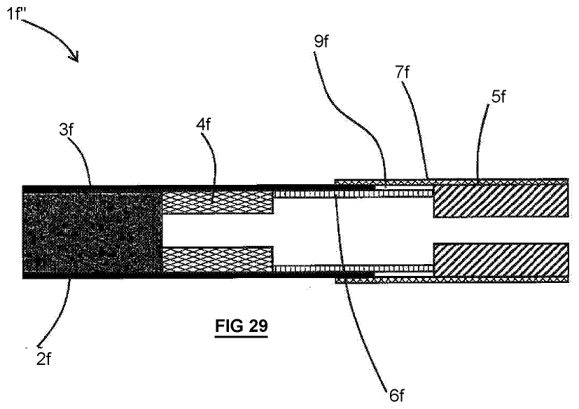

FIG. 29 shows a third embodiment of a consumable 1f which is the same as the first embodiment except that the wrapping layer 3f does not completely circumscribe the cardboard tube spacer 6f such that there is an annular gap 9f between the tipping layer 7f and the cardboard tube spacer 6f downstream of the end of the wrapping layer 3f.

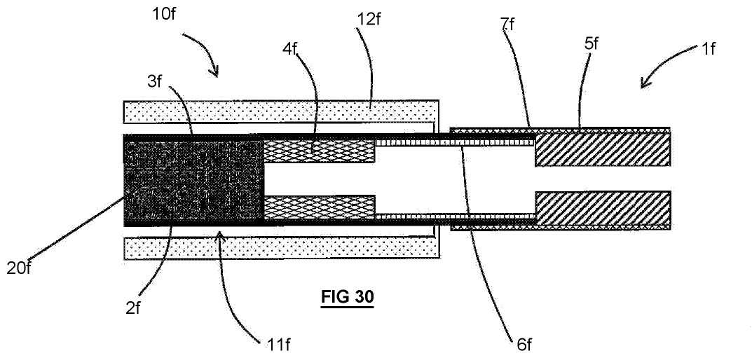

FIG. 30 shows the first embodiment inserted into an HNB device 10f comprising a rod-shaped heating element 20f. The heating element 20f projects into a cavity 11f within the main body 12f of the device.

The consumable 1f is inserted into the cavity 11f of the main body 12f of the device 10f such that the heating rod penetrates the aerosol-forming substrate 2f. Heating of the reconstituted tobacco in the aerosol-forming substrate 2f is effected by powering the heating element (e.g. with a rechargeable battery (not shown)). As the tobacco is heated, moisture and volatile compound (e.g. nicotine) within the tobacco and the aerosol former are released as a vapour and entrained within an airflow generated by inhalation by the user at the terminal filter portion 5f.

As the vapour cools within the upstream filter element 4f and the cardboard tube spacer 6f, it condenses to form an aerosol containing the volatile compounds for inhalation by the user.

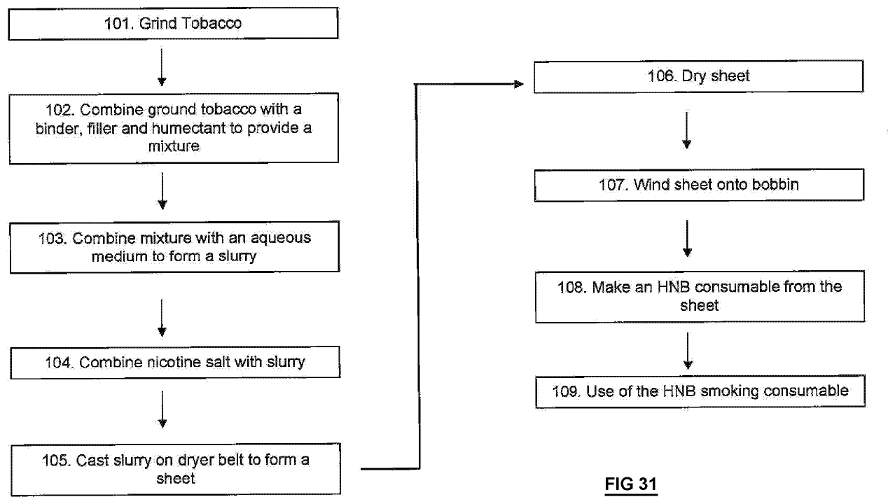

FIG. 31 shows an illustrative diagram of an embodiment of the manufacturing process. Tobacco is ground to reduce the particle size to less than 120 microns 101.

The ground tobacco 101 is then combined with a guar gum binder, cellulose pulp filler, propylene glycol and vegetable glycerine to form a mixture 102.

The mixture 102 is combined with an aqueous medium to form a slurry 103. Mixing of the mixture 102 with the aqueous medium is carried out in a high shear mixer. The tobacco content of the slurry is approximately 50 to 90% of the total solids content of the slurry, where the total solids content is 15 to 30 weight % of the slurry.

A nicotine salt selected from nicotine hydrochloride, nicotine dihydrochloride, nicotine monotartrate, nicotine bitartrate, nicotine bitartrate dihydrate, nicotine benzoate, nicotine fumarate, nictotine orotate, nicotine sulphate, nicotine zinc chloride monohydrate, nicotine lactate and nicotine salicylate, and combinations thereof is combined with the slurry formed in step 103. The amount of nicotine salt is selected so as to ultimately provide a nicotine salt content of 2 to 15 weight % in the dried tobacco sheet.

The resultant slurry 104 is cast onto a dryer belt to form a sheet 105. The sheet is dried using a steam drying apparatus to remove excess moisture content from the sheet until the sheet is self-supporting 106.

The sheet is wound onto a bobbin 107 for storage. The moisture content of the cast sheet at winding is about 7 to 15 percent of dry weight of the tobacco sheet. The sheet can subsequently be made into a whole or part of an HNB consumable suitable for an HNB device 108. This is achieved by cutting the sheet to an appropriate size. The sheet may be used as whole or part of an HNB consumable for an HNB device 109. In particular, it is a slurry-type reconstituted tobacco product for use in an HNB device.

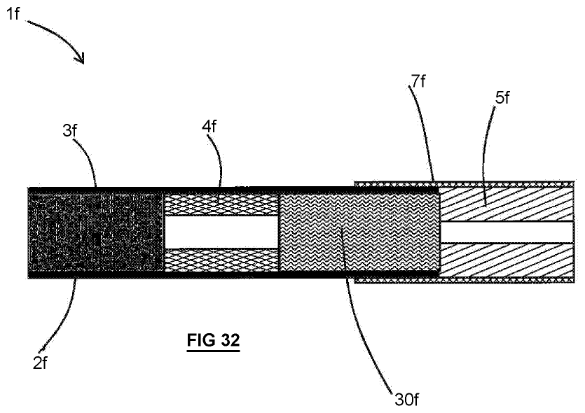

Turning now to consider FIG. 32, there is shown a fourth embodiment of HNB consumable 1f, which is similar in several respects to the first embodiment illustrated in FIG. 27. This embodiment again comprises an aerosol-forming substrate 2f at the upstream end of the consumable 1f. The aerosol-forming substrate comprises reconstituted tobacco which includes nicotine as a volatile compound.

The aerosol-forming substrate 2f comprises 65 wt % tobacco which is provided in the form of gathered shreds produced from a sheet of slurry/paper recon tobacco. The tobacco is dosed with 20 wt % of a humectant such as propylene glycol (PG) or vegetable glycerine (VG) and has a moisture content of between 7-9 wt %. The aerosol-forming substrate further comprises cellulose pulp filler and guar gum binder.

The aerosol-forming substrate 2f is formed in a substantially cylindrical shape such that the consumable resembles a conventional cigarette. It has diameter of around 7 mm and an axial length of around 12 mm.

The aerosol-forming substrate 2f is circumscribed by a paper wrapping layer 3f.

The consumable 1f comprises an upstream filter element 4f and a downstream (terminal) filter element 5f. In this embodiment, the two filter elements 4f, 5f are spaced by a cooling element 30f instead of by the cardboard tube spacer 6f of the previous embodiments.

The cooling element 30f is formed of a gathered sheet of polylactic acid (PLA) which is sprayed with an aqueous solution of a nicotine salt prior to gathering. The gathered sheet provides a cooling element 30f having numerous axially oriented channels which provide a large surface area over which the vapour produced from the aerosol-forming substrate can flow.

Both filter elements 4f, 5f are formed of cellulose acetate tow and wrapped with a respective paper plug layer (not shown).

Both filter elements 4f, 5f have a substantially cylindrical shape. The diameter of the upstream filter 4f matches the diameter of the aerosol-forming substrate 2f. The diameter of the terminal filter element 5f is slightly larger and matches the combined diameter of the aerosol-forming substrate 2f and the wrapping layer 3f. The upstream filter element is slightly shorter in axial length than the terminal filter element 5f at an axial length of 10 mm compared to 12 mm for the terminal filter element 5f.

The cooling element 30f is longer than each of the two filter portions having an axial length of around 14 mm.

Each filter element 4f, 5f is a hollow bore filter element with a hollow, longitudinally extending bore. The diameter of the bore in the upstream filter 4f is slightly larger than the diameter of the bore in the terminal filter 5f, having a diameter of 3 mm compared to 2 mm for the terminal filter element 5f.

The cooling element 30f and the upstream filter portion 4f are circumscribed by the wrapping layer 3f.

The terminal filter element 5f is joined to the upstream elements forming the consumable by a circumscribing paper tipping layer 7f. The tipping layer 7f encircles the terminal filter portion 5f and has an axial length of around 20 mm such that it overlays a portion of the cooling element 30f.

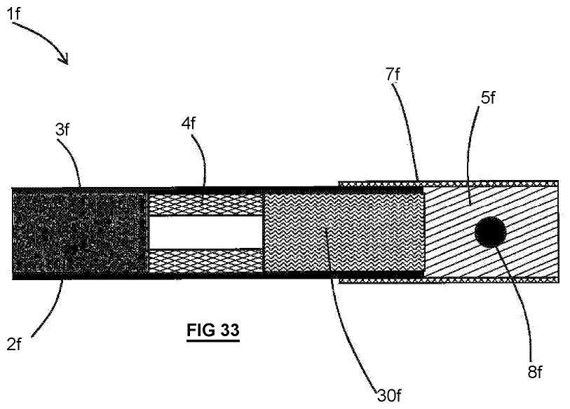

FIG. 33 shows a fifth embodiment of a consumable 1f which is the same as that shown in FIG. 32 except that the terminal filter element 5f is a solid filter element and comprises a crushable capsule 8f (crush-ball) having a shell wall containing a liquid menthol or cherry or vanilla flavourant. The capsule 8f is spherical and has a diameter of 3.5 mm. It is positioned within the axial centre of the terminal filter portion 5f.

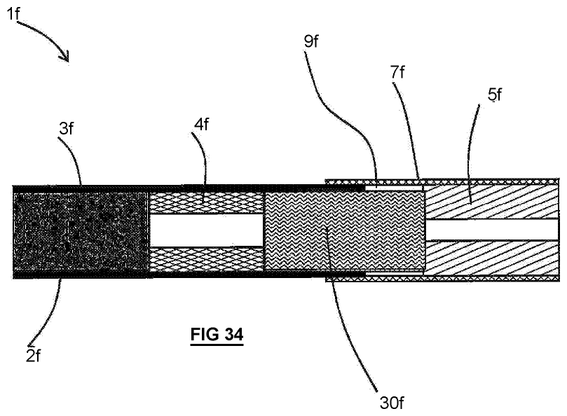

FIG. 34 shows a sixth embodiment of a consumable 1f which is the same as the fourth embodiment except that the wrapping layer 3f does not completely circumscribe the cooling element 30f such that there is an annular gap 9f between the tipping layer 7f and the cooling element 30f downstream of the end of the wrapping layer 3f.

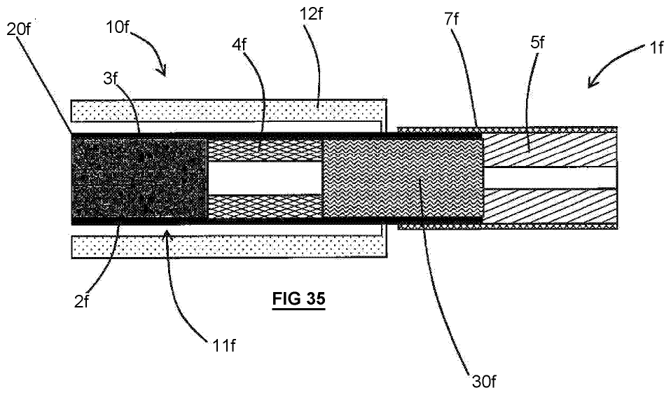

FIG. 35 shows the fourth embodiment inserted into an HNB device 10f comprising a rod-shaped heating element 20f. The heating element 20f projects into a cavity 11f within the main body 12f of the device.

The consumable 1f is inserted into the cavity 11f of the main body 12f of the device 101 such that the heating rod 20f penetrates the aerosol-forming substrate 2f. Heating of the reconstituted tobacco in the aerosol-forming substrate 2f is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter portion 5f.

As the vapour passes through the cooling element 301, heat exchange between the vapour and the cooling element vaporises the nicotine salts and cools the vapour such that condenses to form an aerosol containing the volatile compounds released from both the aerosol-forming substrate 2f and the cooling element 30f for inhalation by the user.

Eighth Mode of the Disclosure: Adaption Providing Greater Ease of Use.

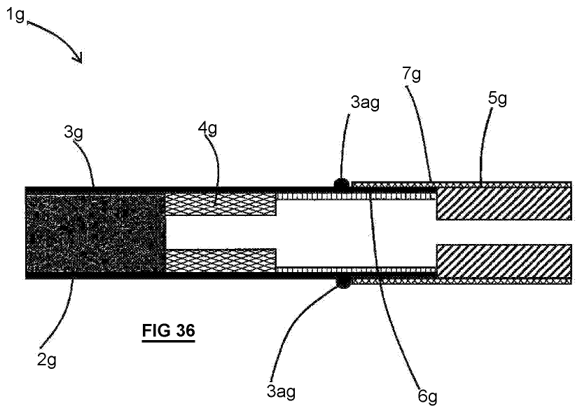

As shown in FIG. 36, the HNB consumable 1g comprises an aerosol-forming substrate 2g at the upstream end of the consumable 1g.

The aerosol-forming substrate 2g comprises reconstituted tobacco which includes nicotine as a volatile compound.

The aerosol-forming substrate 2g comprises 65 wt % tobacco which is provided in the form of gathered shreds produced from a sheet of slurry/paper recon tobacco. The tobacco is dosed with 20 wt % of a humectant such as propylene glycol (PG) or vegetable glycerine (VG) and has a moisture content of between 7-9 wt %. The aerosol-forming substrate further comprises cellulose pulp filler and guar gum binder.

The aerosol-forming substrate 2g is formed in a substantially cylindrical shape such that the consumable resembles a conventional cigarette. It has diameter of around 7 mm and an axial length of around 12 mm.

The aerosol-forming substrate 2g is circumscribed by a paper wrapping layer 3g.

The paper wrapping layer 3g includes a radially- and circumferentially-extending projection which is an annular collar 3ag. The external raised feature 3ag is an integral part of the paper wrapping layer 3g, i.e. the paper wrapping layer 3g is embossed to provide an increased thickness at a specific location thereby providing the collar 3ag.

The consumable 1g comprises an upstream filter element 4g and a downstream (terminal) filter element 5g. The two filter elements 4g, 5g are spaced by a cardboard tube spacer 6g. Both filter elements 4g, 5g are formed of cellulose acetate tow and wrapped with a respective paper plug layer (not shown).

Both filter elements 4g, 5g have a substantially cylindrical shape. The diameter of the upstream filter 4g matches the diameter of the aerosol-forming substrate 2g. The diameter of the terminal filter element 5g is slightly larger and matches the combined diameter of the aerosol-forming substrate 2g and the wrapping layer 3g. The upstream filter element is slightly shorter in axial length than the terminal filter element at an axial length of 10 mm compared to 12 mm for the terminal filter element.

The cardboard tube spacer is longer than each of the two filter elements having an axial length of around 14 mm.

Each filter element 4g, 5g is a hollow bore filter element with a hollow, longitudinally extending bore. The diameter of the bore in the upstream filter is slightly larger than the diameter of the bore in the terminal filter having a diameter of 3 mm compared to 2 mm for the terminal filter element.

The cardboard tube spacer 6g and the upstream filter element 4g are circumscribed by the wrapping layer 3g.

The terminal filter element 5g is joined to the upstream elements forming the consumable by a circumscribing paper tipping layer 7g. The tipping layer 7g encircles the terminal filter element 5g and has an axial length of around 20 mm such that it overlays a portion of the cardboard tube spacer 6g.

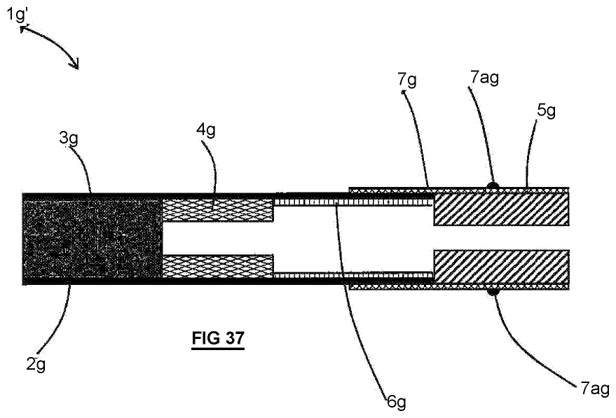

FIG. 37 shows another consumable 1g′ similar to the embodiment of FIG. 36, except that an external raised feature 7ag is present on the paper tipping layer 7g, rather than the paper wrapping layer 3g.

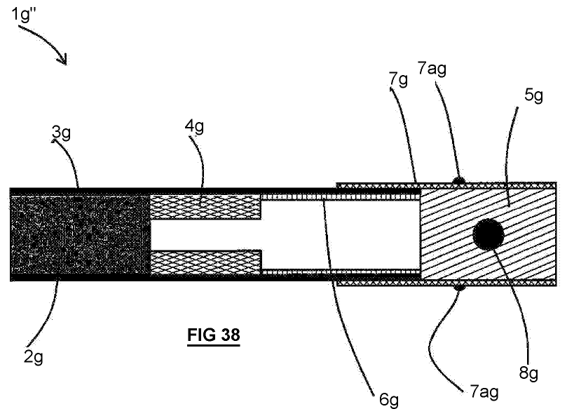

FIG. 38 shows a third embodiment of a consumable 1g″ which is the same as that shown in FIG. 37 except that the terminal filter element 5g is a solid filter element and comprises a crushable capsule 8g (crush-ball) having a shell wall containing a liquid menthol or cherry or vanilla flavourant. The capsule 8g is spherical and has a diameter of 3.5 mm. It is positioned within the axial centre of the terminal filter element 5g.

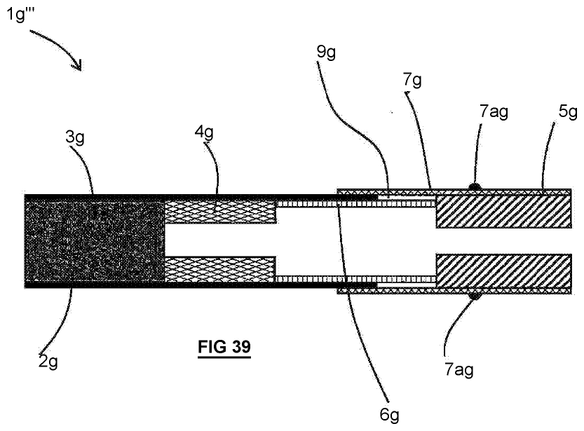

FIG. 39 shows a fourth embodiment of a consumable 1g′″ which is the same as the second embodiment except that the wrapping layer 3g does not completely circumscribe the cardboard tube spacer 6g such that there is an annular gap 9g between the tipping layer 7g and the cardboard tube spacer 6g downstream of the end of the wrapping layer 3g.

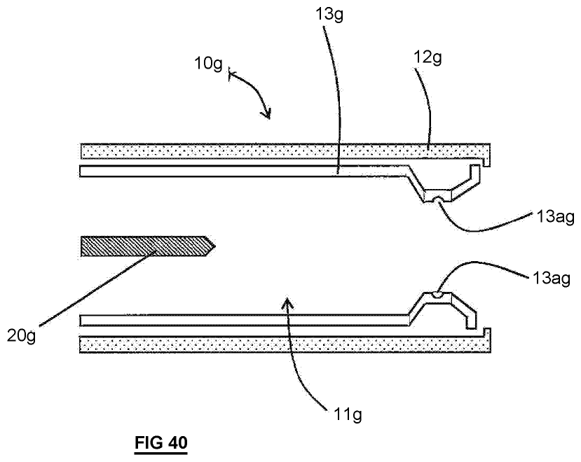

FIG. 40 shows a simplified cross section of an HNB device 10g comprising a main body 12g and an internal annular housing 13g. The housing 13g includes an annular recess 13ag which extends around the entire inner circumference of an inner surface of the housing 13g. The HNB device 10g also includes a rod-shaped heating element 20g which projects into a cavity 11g within the housing 13g of the device.

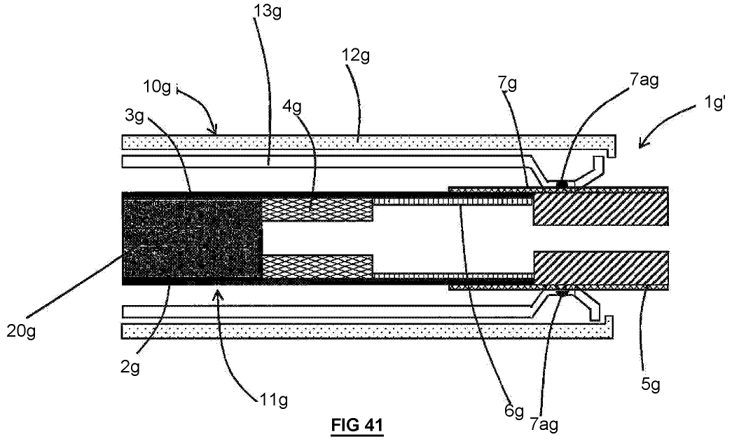

FIG. 41 shows the second embodiment of the HNB consumable 1g′ inserted into the HNB device 10g. The annular collar 3ag on the outer surface of the consumable 1g is engaged with the annular recess 13ag within the internal housing 13g of the HNB device 10g. Movement of the consumable 1g′ within the device 10g is thereby limited, since further insertion or inadvertent removal is made more difficult. After use, the consumable 1g′ may still be removed for disposal by applying enough force to the consumable to overcome the engagement between the raised feature 3ag and the recess 13ag.

The consumable 1g′ is inserted into the cavity 11g of the main body 12g of the device 10g such that the heating rod 20g penetrates the aerosol-forming substrate 2g. Heating of the reconstituted tobacco in the aerosol-forming substrate 2g is effected by powering the heating element (e.g. with a rechargeable battery (not shown)). As the tobacco is heated, moisture and volatile compound (e.g. nicotine) within the tobacco and the humectant are released as a vapour and entrained within an airflow generated by inhalation by the user at the terminal filter element 5g.

As the vapour cools within the upstream filter element 4g and the cardboard tube spacer 6g, it condenses to form an aerosol containing the volatile compounds for inhalation by the user.

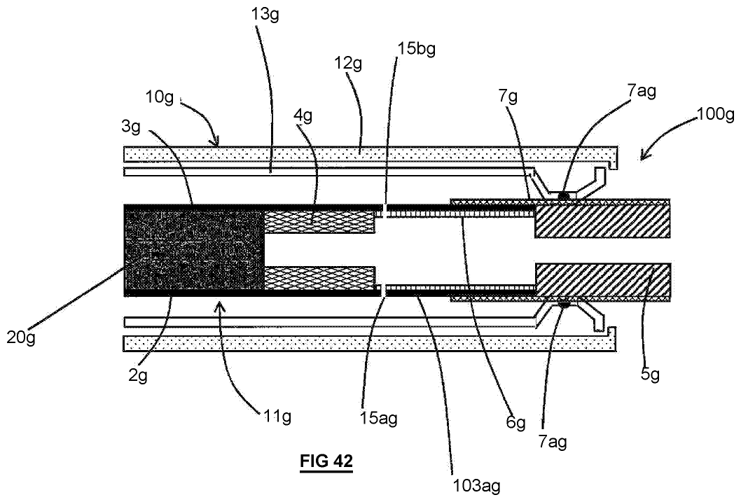

FIG. 42 shows an alternative embodiment of a HNB consumable 100g inserted into the HNB device 10g. The consumable 100g is identical to consumable 1g′ shown in FIGS. 37 and 41, except that the consumable includes ventilation apertures 15ag, 15bg through the paper wrapping layer 3g and the cardboard tube spacer 6g, such that the internal cavity/chamber of the consumable is in fluid communication with the outside of the consumable through the apertures. Two apertures are visible in the cross-section of FIG. 42, which are part of an annular array of ventilation apertures around the entire circumference of the consumable.

During use, as the user draws on the mouthpiece (terminal filter element 5g), a first stream of air passes into the upstream axial end of the consumable, through the aerosol-forming substrate 2g where it collects volatile substances (e.g. nicotine and humectant) and through the continuous channel formed by the hollow bore filter 4g, cardboard tube spacer 6g and terminal hollow bore filter element 5g. A second stream of air passes between the internal surface of HNB device housing 13g and the external surface of the consumable, through the ventilation apertures 15ag, 15bg and other apertures in the annular array (not shown), into the chamber within the consumable defined by the cardboard tube spacer 6g. The second stream combines with the first stream within this chamber before passing through the terminal hollow bore filter element 5g and into the mouth of the user. The ventilation apertures 15ag, 15bg therefore provide additional ventilation which helps to cool the vapour to form the aerosol, encourages more homogeneous mixing of the smoke ingredients and may be used to provide a more desirable resistance to draw for the user. More controlled ventilation through the apertures 15ag, 15bg is achieved by providing a relatively air-tight seal between the annular collar 7ag and the annular recess 13ag.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the disclosure has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. An aerosol-forming article comprising an aerosol-forming substrate wherein the aerosol-forming substrate comprises a pre-formed axially-extending recess adapted to receive an external heating element.

2. An article according to claim 1 wherein the article is a heat not burn consumable.

3. An article according to claim 1 wherein the axially-extending recess has an upstream axial end and a downstream axial end wherein the downstream axial end of spaced from the downstream axial end of the substrate.

4. An article according to claim 3 wherein the axial spacing between the upstream and downstream axial ends of the axially-extending recess is between 1 and 7 mm.

5. An article according to claim 1 wherein the axially-extending recess has a substantially circular radial cross-section at its upstream axial end with a diameter of between 1 and 3 mm.

6. An article according to claim 1 wherein the axially-extending recess has a substantially uniform radial cross-sectional area along its axial length.

7. An article according to claim 1 wherein the axially-extending recess tapers to a reduced cross-sectional area/diameter at a downstream axial end.

8. An article according to claim 1 further comprising a filter element downstream and axially adjacent the aerosol-forming substrate.

9. An article according to claim 8 further comprising a terminal mouth filter element at the axial downstream end of the article.

10. An article according to claim 9 wherein the filter element and terminal filter element are axially spaced by a cooling element or spacer.

11. An article according to claim 8 wherein the filter element and/or the terminal filter element is/are a hollow bore element.

12. A smoking substitute system comprising an article according to claim 1 and a device comprising an elongated heating element.

13. A method of using the system according to claim 12, the method comprising:
inserting the article into the device such that the heating element is received in the axially extending recess; and
heating the article using the heating element.

14.-126. (canceled)
